(19) United States
(12) Patent Application Publication
(10) Pub. No.: US 2026/0053743 A1
(43) Pub. Date: Feb. 26, 2026

(54) TOPICAL SKIN FORMULATIONS OF A PHARMACEUTICALLY ACCEPTABLE SALT OF RUXOLITINIB

(71) Applicant: INCYTE CORPORATION, Wilmington, DE (US)

(72) Inventors: Russell ELLIOTT, Wilmington, DE (US); Zhongjiang JIA, Wilmington, DE (US); Gary LAWRENCE, Wilmington, DE (US); Mei LI, Wilmington, DE (US); Pingli LIU, Wilmington, DE (US); Weiguo LIU, Wilmington, DE (US); David MELONI, Wilmington, DE (US); Yongchun PAN, Wilmington, DE (US); Huifang WU, Wilmington, DE (US); Jiacheng ZHOU, Wilmington, DE (US)

(21) Appl. No.: 19/308,662

(22) Filed: Aug. 25, 2025

Related U.S. Application Data
(60) Provisional application No. 63/687,224, filed on Aug. 26, 2024.
(60) Provisional application No. 63/688,052, filed on Aug. 28, 2024.

(51) Int. Cl. A61K 9/107; A61K 9/00; A61K 9/06; A61K 31/519; A61K 47/02; A61K 47/10; A61K 47/14; A61K 47/18; A61K 47/26; A61K 47/34; A61K 47/36

(52) U.S. Cl. CPC A61K 9/107; A61K 9/0014; A61K 9/06; A61K 31/519; A61K 47/02; A61K 47/10; A61K 47/14; A61K 47/183; A61K 47/26; A61K 47/34; A61K 47/36

(57) ABSTRACT

This invention relates to topical skin formulations comprising a pharmaceutically acceptable salt of ruxolitinib, and use in the treatment of skin disorders.

This application claims the benefit of priority of U.S. Provisional Application No. 63/687,224, filed Aug. 26, 2024, and U.S. Provisional Application No. 63/688,052, filed Aug. 28, 2024, each of which is incorporated herein by reference in its entirety.

### TECHNICAL FIELD

This invention relates to topical skin formulations comprising a pharmaceutically acceptable salt of ruxolitinib, with improved stability, and their use in the treatment of skin disorders.

### BACKGROUND

Protein kinases (PKs) regulate diverse biological processes including cell growth, survival, differentiation, organ formation, morphogenesis, neovascularization, tissue repair, and regeneration, among others. Protein kinases also play specialized roles in a host of human diseases including cancer. Cytokines, low-molecular weight polypeptides or glycoproteins, regulate many pathways involved in the host inflammatory response to sepsis. Cytokines influence cell differentiation, proliferation and activation, and can modulate both pro-inflammatory and anti-inflammatory responses to allow the host to react appropriately to pathogens. Signaling of a wide range of cytokines involves the Janus kinase family (JAKs) of protein tyrosine kinases and Signal Transducers and Activators of Transcription (STATs). There are four known mammalian JAKs: JAK1 (Janus kinase-1), JAK2, JAK3 (also known as Janus kinase, leukocyte; JAKL; and L-JAK), and TYK2 (protein-tyrosine kinase 2).

Cytokine-stimulated immune and inflammatory responses contribute to pathogenesis of diseases: pathologies such as severe combined immunodeficiency (SCID) arise from suppression of the immune system, while a hyperactive or inappropriate immune/inflammatory response contributes to the pathology of autoimmune diseases (e.g., asthma, systemic lupus erythematosus, thyroiditis, myocarditis), and illnesses such as scleroderma and osteoarthritis (Ortmann, R. A., T. Cheng, et al. (2000) Arthritis Res 2 (1): 16-32).

Deficiencies in expression of JAKs are associated with many disease states. For example, Jak1−/− mice are runted at birth, fail to nurse, and die perinatally (Rodig, S. J., M. A. Meraz, et al. (1998) Cell 93 (3): 373-83). Jak2−/− mouse embryos are anemic and die around day 12.5 postcoitum due to the absence of definitive erythropoiesis.

The JAK/STAT pathway, and in particular all four JAKs, are believed to play a role in the pathogenesis of asthmatic response, chronic obstructive pulmonary disease, bronchitis, and other related inflammatory diseases of the lower respiratory tract. Multiple cytokines that signal through JAKs have been linked to inflammatory diseases/conditions of the upper respiratory tract, such as those affecting the nose and sinuses (e.g., rhinitis and sinusitis) whether classically allergic reactions or not. The JAK/STAT pathway has also been implicated in inflammatory diseases/conditions of the eye and chronic allergic responses.

Activation of JAK/STAT in cancers may occur by cytokine stimulation (e.g. IL-6 or GM-CSF) or by a reduction in the endogenous suppressors of JAK signaling such as SOCS (suppressor or cytokine signaling) or PIAS (protein inhibitor of activated STAT) (Boudny, V., and Kovarik, J., Neoplasm. 49:349-355, 2002). Activation of STAT signaling, as well as other pathways downstream of JAKs (e.g., Akt), has been correlated with poor prognosis in many cancer types (Bowman, T., et al. Oncogene 19:2474-2488, 2000). Elevated levels of circulating cytokines that signal through JAK/STAT play a causal role in cachexia and/or chronic fatigue. As such, JAK inhibition may be beneficial to cancer patients for reasons that extend beyond potential anti-tumor activity.

Inhibition of the JAK kinases is also envisioned to have therapeutic benefits in patients suffering from skin immune disorders such as psoriasis, and skin sensitization. In psoriasis vulgaris, the most common form of psoriasis, it has been generally accepted that activated T lymphocytes are important for the maintenance of the disease and its associated psoriatic plaques (Gottlieb, A. B., et al, Nat Rev Drug Disc., 4:19-34). Psoriatic plaques contain a significant immune infiltrate, including leukocytes and monocytes, as well as multiple epidermal layers with increased keratinocyte proliferation. While the initial activation of immune cells in psoriasis occurs by an ill defined mechanism, the maintenance is believed to be dependent on a number of inflammatory cytokines, in addition to various chemokines and growth factors (JCI, 113:1664-1675). Many of these, including interleukins −2, −4, −6, −7, −12, −15, −18, and −23 as well as GM-CSF and IFNg, signal through the Janus (JAK) kinases (Adv Pharmacol. 2000; 47:113-74). As such, blocking signal transduction at the level of JAK kinases may result in therapeutic benefits in patients suffering from psoriasis or other immune disorders of the skin.

Ruxolitinib phosphate cream (1.5% w/w) (OPZELURA®) is approved as a topical skin formulation for treatment of atopic dermatitis and non-segmental vitiligo. Recently, it was discovered that ruxolitinib dihydrate crystals may form at larger batch sizes of ruxolitinib cream. Hence, there is a need to develop improved formulations of ruxolitinib cream which negate the appearance of ruxolitinib dihydrate crystals during processing at large scale. The formulations of the invention are directed toward this need and other ends.

### SUMMARY

Ruxolitinib is a potent JAK1/JAK2 inhibitor. Its chemical name is (R)-3-(4-(7H-pyrrolo[2,3-d]pyrimidin-4-yl)-1H-pyrazol-1-yl)-3-cyclopentylpropanenitrile. Ruxolitinib and its pharmaceutically acceptable salts have previously been described in U.S. Pat. Nos. 7,598,257, 8,722,693, and 11,905,292, each of which is incorporated herein by reference in its entirety. The present invention describes ruxolitinib and pharmaceutically acceptable salts thereof suitable for topical administration and treatment of skin disorders.

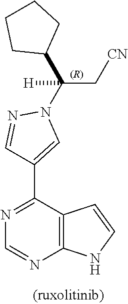

Accordingly, the present invention provides, inter alia, a topical skin formulation, wherein the topical formulation is an oil-in-water emulsion, comprising:
about 0.5% to about 1.5% of a pharmaceutically acceptable salt of ruxolitinib on a free base basis by weight of the formulation;
an aqueous component;
an oil component
an non-ionic emulsifier component; and
a pharmaceutically acceptable acid.

The present invention also provides a method of treating a skin disorder, comprising applying a topical skin formulation described herein to an area of skin of the patient.

The present invention also provides a topical skin formulation described herein for use in treatment of a skin disorder in a patient in need thereof.

The present invention also provides use of a topical skin formulation described herein for the preparation of a medicament for use in treatment of a skin disorder in a patient in need thereof.

In other embodiments, the present invention provides a process of making the topical skin formulations of the disclosure.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 depicts a microscopy image showing rod-like ruxolitinib dihydrate crystals as observed in the cream formulation.

FIG. 2 depicts a microscopy image showing the growth of ruxolitinib dihydrate crystals at the oil droplet interface in the cream formulation.

FIG. 3A shows the raw microscopy image for the Example 3 formulation.

FIG. 3B shows the raw microscopy image for the original formulation without added phosphoric acid.

FIG. 3C shows the microscopy image in FIG. 3A after IMAGIC image analysis.

FIG. 3D shows the microscopy image in FIG. 3B after IMAGIC image analysis.

FIG. 4A depicts a histogram showing the globule size distribution for the Example 3 formulation.

FIG. 4B depicts a histogram showing the globule size distribution for the original formulation without added phosphoric acid.

FIG. 5 depicts the results from a stability study using a LUMiSizer® Dispersion Analyzer for the Example 3 formulation and the original formulation without phosphoric acid added.

### DETAILED DESCRIPTION

Ruxolitinib can be prepared as described in U.S. Pat. Nos. 7,598,257 and 11,905,292, each of which is incorporated herein by reference in its entirety. The 1:1 phosphate, sulfate, and maleate salts of ruxolitinib can be prepared as described in U.S. Pat. Nos. 8,722,693, and 11,905,292, each of which is incorporated herein by reference in its entirety. The oxalate, L-tartrate, and hydrochloric acid salts of ruxolitinib can be prepared as described in WO 2016063294, which is incorporated herein by reference in its entirety. The mesylate, ediyslate, napadisylate, and acesulfmate salts of ruxolitinib can be prepared as described in WO 2023087101 which is incorporated herein by reference in its entirety. The hydrobromic and hydroiodate salts of ruxolitinib can be prepared as described in WO2017008772, which is incorporated herein by reference in its entirety.

Recently, it was discovered that ruxolitinib dihydrate crystals may form at larger batch sizes of ruxolitinib cream (e.g., 3200 kg, 900 kg). Ruxolitinib dihydrate can be characterized as summarized in US20230399331, which is herein incorporated in its entirety. It has been unexpectedly discovered that addition of a pharmaceutically acceptable acid can eliminate the appearance of ruxolitinib dihydrate crystals in the topical skin formulations over batches without such acid.

Without wishing to be bound by any particular theory, it is believed that pharmaceutically acceptable salts of ruxolitinib, such as ruxolitinib phosphate can dissociate in water to ruxolitinib free base and the ruxolitinib salt (e.g., ruxolitinib phosphate). Ruxolitinib free base may associate on the surface of larger emulsion droplets in the formulation, leading to further association on the droplet surface, allowing for heterogenous crystal nucleation at the surface. Because ruxolitinib dihydrate is less soluble than salts such as ruxolitinib phosphate (1.57 mg/mL versus 2.06 mg/mL at pH 3), ruxolitinib dihydrate crystals can form. Without wishing to be bound by any particular theory, the addition of a pharmaceutically acceptable acid is believed to reduce the amount of ruxolitinib free base available for accumulation at the interface of emulsion droplet by driving the equilibrium towards the protonated ruxolitinib molecule, thereby eliminating the appearance of ruxolitinib dihydrate crystals in the topical skin formulations.

Provided herein is a topical skin formulation, wherein the topical formulation is an oil-in-water emulsion, comprising:
about 0.5% to about 1.5% of a pharmaceutically acceptable salt of ruxolitinib on a free base basis by weight of the formulation;
an aqueous component;
an oil component
an non-ionic emulsifier component; and
a pharmaceutically acceptable acid.

In some embodiments, the pharmaceutically acceptable acid is selected from phosphoric acid, citric acid, and lactic acid.

In some embodiments, the pharmaceutically acceptable acid is phosphoric acid. In some embodiments, the phosphoric acid is present in an amount of about 0.05% to about 0.5% by weight of the formulation. In some embodiments, the pharmaceutically acceptable acid is phosphoric acid. In some embodiments, the phosphoric acid is present in an amount of about 0.1% to about 0.5% by weight of the formulation. In some embodiments, the phosphoric acid is present in an amount of about 0.1% to about 0.3% by weight of the formulation. In some embodiments, the phosphoric acid is present in an amount of about 0.15% to about 0.25% by weight of the formulation. In some embodiments, the phosphoric acid is present in an amount of about 0.2% by weight of the formulation. In some embodiments, the phosphoric acid is present in an amount of about 0.05% to about 0.1% by weight of the formulation. In some embodiments, the phosphoric acid is present in an amount of about 0.05% to about 0.15% by weight of the formulation. In some embodiments, the phosphoric acid is present in an amount of about 0.05% to about 0.2% by weight of the formulation.

In some embodiments, the pharmaceutically acceptable acid is citric acid or lactic acid. In some embodiments, the citric acid or lactic acid is present in an amount of about 1% to about 5% by weight of the formulation. In some embodiments, the citric acid or lactic acid is present in an amount of about 2% to about 5% by weight of the formulation. In some embodiments, the citric acid or lactic acid is present in an amount of about 3% to about 5% by weight of the formulation.

The topical skin formulations of the disclosure are suitable for administration to the skin of a human in order to treat skin diseases.

In some embodiments, the formulation is a cream.

In some embodiments, the formulation is a solubilized cream.

In some embodiments, there is an absence of ruxolitinib dihydrate in the topical skin formulation. In some embodiments, the oil component comprises from about 10% to about 30% by weight of the formulation. In some embodiments, the oil component comprises from about 15% to about 30% by weight of the formulation. In some embodiments, the oil component comprises from about 20% to about 27% by weight of the formulation.

In some embodiments, the oil component comprises from about 20% to about 25% by weight of the formulation.

In some embodiments, the aqueous component comprises from about 40% to about 90% by weight of the formulation. In some embodiments, the aqueous component comprises from about 50% to about 80% by weight of the formulation. In some embodiments, the aqueous component comprises from about 60% to about 80% by weight of the formulation. In some embodiments, the aqueous component comprises from about 40% to about 60% of water by weight of the formulation. In some embodiments, the aqueous component comprises from about 40% to about 60% of water by weight of the formulation. In some embodiments, the aqueous component comprises from about 45% to about 55% of water by weight of the formulation.

In some embodiments, the non-ionic emulsifier component is present in an amount of about 2% to about 6% by weight of the formulation. In some embodiments, the non-ionic emulsifier component is present in an amount of about 3% to about 5% by weight of the formulation. In some embodiments, the non-ionic emulsifier component is present in an amount of about 4% to about 5% by weight of the formulation.

In some embodiments, the topical skin formulation comprises (by weight of the formulation):
about 10% to about 30% of the oil component;
about 40% to about 90% of the aqueous component;
about 0.5% to about 1.5% of the pharmaceutically acceptable salt of ruxolitinib; and
about 0.1% to about 0.5% of phosphoric acid.

In some embodiments, the topical skin formulation comprises (by weight of the formulation):
about 15% to about 30% of the oil component;
about 50% to about 80% of the aqueous component;
about 0.5% to about 1.5% of the pharmaceutically acceptable salt of ruxolitinib; and
about 0.1% to about 0.5% of phosphoric acid.

In some embodiments, the topical skin formulation comprises (by weight of the formulation):
about 20% to about 27% of the oil component;
about 60% to about 80% of the aqueous component;
about 0.5% to about 1.5% of the pharmaceutically acceptable salt of ruxolitinib; and
about 0.1% to about 0.5% of phosphoric acid.

In some embodiments, the topical skin formulation comprises (by weight of the formulation):
about 10% to about 30% of the oil component;
about 40% to about 90% of the aqueous component;
about 0.5% to about 1.5% of the pharmaceutically acceptable salt of ruxolitinib; and
about 0.05% to about 0.15% of phosphoric acid.

In some embodiments, the topical skin formulation comprises (by weight of the formulation):
about 15% to about 30% of the oil component;
about 50% to about 80% of the aqueous component;
about 0.5% to about 1.5% of the pharmaceutically acceptable salt of ruxolitinib; and
about 0.05% to about 0.1% of phosphoric acid.

In some embodiments, the topical skin formulation comprises (by weight of the formulation):
about 20% to about 27% of the oil component;
about 60% to about 80% of the aqueous component;
about 0.5% to about 1.5% of the pharmaceutically acceptable salt of ruxolitinib; and
about 0.05% to about 0.1% of phosphoric acid.

In each of the preceding embodiments, the formulation comprises about 40% to about 60% of water by weight of the formulation. In each of the preceding embodiments, the aqueous component comprises about 45% to about 50% of water by weight of the formulation. In each of the preceding three embodiments, the formulation comprises about 40% to about 60% of water by weight of the formulation.

In some embodiments, the oil component comprises one or more substances independently selected from petrolatums, fatty alcohols, mineral oils, triglycerides, and silicone oils.

In some embodiments, the oil component comprises one or more substances independently selected from white petrolatum, cetyl alcohol, stearyl alcohol, light mineral oil, medium chain triglycerides, and dimethicone 350.

In some embodiments, the oil component comprises one or more petrolatums, one or more fatty alcohols, one or more mineral oils, one or more triglycerides, and one or more silicone oils.

In some embodiments, the oil component comprises white petrolatum, cetyl alcohol, stearyl alcohol, light mineral oil, medium chain triglycerides, and dimethicone 350.

In some embodiments, the oil component comprises, by weight of the formulation:
about 3% to about 5% of one or more mineral oils;
about 6% to about 8% of one or more petrolatums;
about 4% to about 6% of one or more fatty alcohols;
about 0.5% to about 1.5% of one of more silicone oils; and
about 4% to about 6% of one or more trigylcerides.

In some embodiments, the oil component comprises, by weight of the formulation:
about 3% to about 5% of light mineral oil;
about 6% to about 8% of white petrolatum;
about 4% to about 6% of a mixture of cetyl alcohol and stearyl alcohol;
about 0.5% to about 1.5% of dimethicone 350; and
about 4% to about 6% of medium chain trigylcerides.

In some embodiments, the oil component comprises, by weight of the formulation:
about 4% of light mineral oil;
about 7% of white petrolatum;
about 3% of cetyl alcohol;
about 1.75% of stearyl alcohol;
about 1% of dimethicone 350; and
about 5% of medium chain trigylcerides.

In some embodiments, the non-ionic emulsifier component comprises one or more substances independently selected from glyceryl fatty esters and sorbitan fatty esters. In some embodiments, the non-ionic emulsifier component comprises one or more substances independently selected from glyceryl stearate (i.e., glyceryl monostearate), and polysorbate 20. In some embodiments, the non-ionic non-ionic emulsifier component comprises one or more glyceryl fatty esters and one or more sorbitan fatty esters. In some embodiments, the non-ionic emulsifier component comprises glyceryl stearate, and polysorbate 20.

In some embodiments, the non-ionic emulsifier component is present in an amount of about 2% to about 6% by weight of the formulation. In some embodiments, the non-ionic emulsifier component is present in an amount of about 3% to about 5% by weight of the formulation. In some embodiments, the non-ionic emulsifier component is present in an amount of about 4% to about 5% by weight of the formulation. In some embodiments, the non-ionic emulsifier component is present in an amount of about 2.5% to about 3.5% of glyceryl stearate by weight of the formulation and about 1% to about 1.5% % of polysorbate 20 by weight of the formulation. In some embodiments, the non-ionic emulsifier component is present in an amount of about 3% of glyceryl stearate by weight of the formulation and about 1.25% of polysorbate 20 by weight of the formulation.

In some embodiments, the oil component and the non-ionic emulsifier component are combined into an oil phase.

In some embodiments, the aqueous component comprises water and one or more substances independently selected from C₁₋₃ alkyl parabens, alkylene glycols, polyalkylene glycols; polysaccharides, and chelating agents. In some embodiments, the aqueous component comprises water, one or more C₁₋₃ alkyl parabens, one or more alkylene glycols, one or more polyalkylene glycols; one or more polysaccharides, and one or more chelating agents. In some embodiments, the aqueous component comprises water and one or more substances independently selected from methyl paraben, propyl paraben, propylene glycol, polyethylene glycol 200, xanthan gum, and edetate disodium. In some embodiments, the aqueous component comprises water, methyl paraben, propyl paraben, propylene glycol, polyethylene glycol 200, xanthan gum, and edetate disodium.

In some embodiments, the aqueous component comprises, by weight of the formulation:
about 40% to about 60% water;
about 10 to about 20% of one or more alkylene glycols;
about 5% to about 10% of one or more polyalkylene glycols;
about 0.03% to about 0.2% of one or more C₁₋₃ alkyl parabens;
about 0.3% to about 0.5% of one or more polysaccharides; and
about 0.01% to about 0.1% of one or more chelating agents.

In some embodiments, the aqueous component comprises, by weight of the formulation:
about 45% to about 50% water;
about 13 to about 17% of one or more alkylene glycols;
about 6% to about 8% of one or more polyalkyelene glycols;
about 0.05% to about 0.2% of one or more C₁₋₃ alkyl parabens;
about 0.3% to about 0.5% of one or more polysaccharides; and
about 0.03% to about 0.07% of one or more chelating agents.

In some embodiments, the aqueous component comprises, by weight of the formulation:
about 45% to about 50% water;
about 14 to about 16% of one or more alkylene glycols;
about 6% to about 8% of one or more polyalkyelene glycols;
about 0.05% to about 0.2% of one or more C₁₋₃ alkyl parabens;
about 0.3% to about 0.5% of one or more polysaccharides; and
about 0.04% to about 0.06% of one or more chelating agents.

In some embodiments, the aqueous component comprises, by weight of the formulation:
about 45% to about 50% water;
about 13 to about 17% of propylene glycol;
about 6% to about 8% of polyethylene glycol 200;
about 0.05% to about 0.15% of methyl paraben;
about 0.01% to about 0.1% of propyl paraben;
about 0.3% to about 0.5% of xanthan gum;
about 0.03% to about 0.07% of edetate disodium.

In some embodiments, the aqueous component comprises, by weight of the formulation:
about 45% to about 50% water;
about 14 to about 16% of propylene glycol;
about 6% to about 8% of polyethylene glycol 200;
about 0.08% to about 0.12% of methyl paraben;
about 0.03% to about 0.08% of propyl paraben;
about 0.3% to about 0.5% of xanthan gum; and
about 0.04% to about 0.06% of edetate disodium.

In some embodiments, the aqueous component comprises, by weight of the formulation:
about 45% to about 50% water;
about 15% of propylene glycol;
about 7% of polyethylene glycol 200;
about 0.1% of methyl paraben;
about 0.05% of propyl paraben;
about 0.4% of xanthan gum; and
about 0.05% of edetate disodium.

In some embodiments, the topical skin formulation further comprises about 0.1% to about 0.8% of phenoxyethanol. In some embodiments, the topical skin formulation further comprises about 0.4% to about 0.6% of phenoxyethanol. In some embodiments, the topical skin formulation further comprises about 0.5% of phenoxyethanol.

In some embodiments, the pharmaceutically acceptable salt of ruxolitinib is ruxolitinib phosphate, ruxolitinib chloride, ruxolitinib mesylate, ruxolitinib maleate, ruxolitinib sulfate, ruxolitinib ediyslate, ruxolitinib napadisylate, ruxolitinib acesulfmate, ruxolitinib oxalate, ruxolitinib L-tartrate, ruxolitinib hydrobromide, and ruxolitinib hydroiodate. In some embodiments, the pharmaceutically acceptable salt of ruxolitinib is ruxolitinib phosphate, ruxolitinib chloride, ruxolitinib mesylate, ruxolitinib maleate, or ruxolitinib sulfate. In some embodiments, the pharmaceutically acceptable salt of ruxolitinib is ruxolitinib phosphate. In some embodiments, the pharmaceutically acceptable salt of ruxolitinib is ruxolitinib chloride. In some embodiments, the pharmaceutically acceptable salt of ruxolitinib is ruxolitinib mesylate. In some embodiments, the pharmaceutically acceptable salt of ruxolitinib is ruxolitinib maleate. In some embodiments, the pharmaceutically acceptable salt of ruxolitinib is ruxolitinib sulfate. In some embodiments, the pharmaceutically acceptable salt of ruxolitinib is ruxolitinib ediyslate. In some embodiments, the pharmaceutically acceptable salt of ruxolitinib is ruxolitinib napadisylate. In some embodiments, the pharmaceutically acceptable salt of ruxolitinib is ruxolitinib acesulfmate. In some embodiments, the pharmaceutically acceptable salt of ruxolitinib is ruxolitinib oxalate. In some embodiments, the pharmaceutically acceptable salt of ruxolitinib is ruxolitinib L-tartrate. In some embodiments, the pharmaceutically acceptable salt of ruxolitinib is ruxolitinib hydrobromide. In some embodiments, the pharmaceutically acceptable salt of ruxolitinib is ruxolitinib hydroiodate.

In some embodiments, the pharmaceutically acceptable salt of ruxolitinib is present in an amount of about 0.5% by weight of the formulation on a free base basis. In some embodiments, the pharmaceutically acceptable salt of ruxolitinib is present in an amount of about 0.75% by weight of the formulation on a free base basis. In some embodiments, the pharmaceutically acceptable salt of ruxolitinib is present in an amount of about 1% by weight of the formulation on a free base basis. In some embodiments, the pharmaceutically acceptable salt of ruxolitinib is present in an amount of about 1.5% by weight of the formulation on a free base basis.

In some embodiments, the pH is not greater than 3.6. In some embodiments, the pH is not greater than 3.4. In some embodiments, the pH of the formulation is less than about 3.3. In some embodiments, the pH of the formulation is less than 3.2. In some embodiments, the pH is less than 3.1. In some embodiments, the pH is less than 3.0. In some embodiments, the pH is less than 2.9. In some embodiments, the pH ranges from 2.9 to 3.6. In some embodiments, the pH of the formulation ranges from about 2.6 to about 3.3.

In some embodiments, the topical skin formulation is an oil-in-water emulsion. In some embodiments, the formulation is a cream. In some embodiments, the formulation is a solubilized cream. In some embodiments, the formulation has a droplet size is above about 3 microns. In some embodiments, the droplet size ranges from above about 3 microns to below about 18 microns. In some embodiments, the droplet size ranges from about 4 microns to about 7 microns. In some embodiments, the droplet size is about 6 microns. In some embodiments, the average droplet size distribution is about 50% above 6 microns. In some embodiments, the average droplet size distribution should have about 50% below 6 microns. In some embodiments, the average droplet size distribution is about 10% above 9 microns. In some embodiments, the average droplet size distribution is about 90% below 9 microns. In some embodiments, the average droplet size distribution is at least about 90% above 4 microns.

As used herein, the term “emulsifier component” refers, in one aspect, to a substance, or mixtures of substances that maintains an element or particle in suspension within a fluid medium. In some embodiments, the emulsifier component allows an oil phase to form an emulsion when combined with water. In some embodiments, the emulsifier component refers to one or more non-ionic surfactants.

As used herein, the phrase “chelating agent” refers to a compound or mixtures of compounds that has the ability to bind strongly with metal ions.

As used herein, the term “non-ionic emulsifier component” refers, in one aspect, to a non-ionic substance, or mixtures of non-ionic substances that maintains an element or particle in suspension within a fluid medium. In some embodiments, the non-ionic emulsifier component allows an oil phase to form an emulsion when combined with water. In some embodiments, the non-ionic emulsifier component refers to one or more non-ionic surfactants.

As used herein, the term “pharmaceutically acceptable salt” refers to a salt formed by the addition of a pharmaceutically acceptable acid or base to a compound disclosed herein. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in their entireties.

It will also be understood that compounds described herein may exist in solvated, for example hydrated, as well as unsolvated forms. It will further be understood that the present invention encompasses all such solvated forms of the compounds.

As used herein, “% by weight of the formulation” means the percent concentration of the component in the formulation is on weight/weight basis. For example, 1% w/w of component A=[(mass of component A)/(total mass of the formulation)]×100.

As used herein, “% by weight of the formulation on a free base basis” of ruxolitinib, or pharmaceutically acceptable salt thereof” means that the % w/w is calculated based on the weight of ruxolitinib in the total formulation. For example, “0.5% w/w on a free base basis” of ruxolitinib phosphate means that for 100 grams of total formulation, there are 0.66 grams of ruxolitinib in the formulation (which equates to 0.5 grams of the free base of ruxolitinib.

In some embodiments, the components are present in exactly the ranges specified (e.g., the term “about” is not present). In some embodiments, “about” means plus or minus 10% of the value. In some embodiments, “about” means plus or minus 5% of the value. In some embodiments, “about” means plus or minus 2.5% the value.

As will be appreciated, some components of the topical skin formulations described herein can possess multiple functions. For example, a given substance may act as both an emulsifying agent component and a stabilizing agent. In some such cases, the function of a given component can be considered singular, even though its properties may allow multiple functionality. In some embodiments, each component of the formulation comprises a different substance or mixture of substances.

As used herein, “aqueous component” means a mixtures of substances which includes water and which may include a mixture of other substances soluble or miscible with water. The aqueous component may comprise one or more co-solvents that are capable of solubilizing a pharmaceutically acceptable salt of ruxolitinib.

As used herein, the term “component” can mean one substance or a mixture of substances.

As used herein, the term “fatty acid” refers to an aliphatic acid that is saturated or unsaturated. In some embodiments, the fatty acid is in a mixture of different fatty acids. In some embodiments, the fatty acid has between about eight to about thirty carbons on average. In some embodiments, the fatty acid has about 12 to 20, 14-20, or 16-18 carbons on average. Suitable fatty acids include, but are not limited to, cetyl acid, stearic acid, lauric acid, myristic acid, erucic acid, palmitic acid, palmitoleic acid, capric acid, caprylic acid, oleic acid, linoleic acid, linolenic acid, hydroxystearic acid, 12-hydroxystearic acid, cetostearic acid, isostearic acid, sesquioleic acid, sesqui-9-octadecanoic acid, sesquiisooctadecanoic acid, behenic acid, isobchenic acid, and arachidonic acid, or mixtures thereof.

As used herein, the term “fatty alcohol” refers to an aliphatic alcohol that is saturated or unsaturated. In some embodiments, the fatty alcohol is in a mixture of different fatty alcohols. In some embodiments, the fatty alcohol has between about 12 to about 20, about 14 to about 20, or about 16 to about 18 carbons on average. Suitable fatty alcohols include, but are not limited to, myristyl alcohol, stearyl alcohol, lauryl alcohol, palmityl alcohol, cetyl alcohol, capryl alcohol, caprylyl alcohol, oleyl alcohol, linolenyl alcohol, arachidonic alcohol, behenyl alcohol, isobehenyl alcohol, selachyl alcohol, chimyl alcohol, and linoleyl alcohol, or mixtures thereof.

As used herein, the term “polyalkylene glycol”, employed alone or in combination with other terms, refers to a polymer containing oxyalkylene monomer units, or copolymer of different oxyalkylene monomer units, wherein the alkylene group has 2 to 6, 2 to 4, or 2 to 3 carbon atoms. As used herein, the term “oxyalkylene”, employed alone or in combination with other terms, refers to a group of formula-O-alkylene-. In some embodiments, the polyalkylene glycol is polyethylene glycol.

As used herein, the term, “sorbitan fatty ester” includes products derived from sorbitan or sorbitol and fatty acids and, optionally, poly(ethylene glycol) units, including sorbitan esters and polyethoxylated sorbitan esters. In some embodiments, the sorbitan fatty ester is a polyethoxylated sorbitan ester.

As used herein, the term “sorbitan ester” refers to a compound, or mixture of compounds, derived from the esterification of sorbitol and at least one fatty acid. Fatty acids useful for deriving the sorbitan esters include, but are not limited to, those described herein. Suitable sorbitan esters include, but are not limited to, the Span™ series (available from Uniqema), which includes Span 20 (sorbitan monolaurate), 40 (sorbitan monopalmitate), 60 (sorbitan monostearate), 65 (sorbitan tristearate), 80 (sorbitan monooleate), and 85 (sorbitan trioleate). Other suitable sorbitan esters include those listed in R. C. Rowe and P. J. Shesky, Handbook of pharmaceutical excipients, (2006), 5th ed., which is incorporated herein by reference in its entirety.

As used herein, the term “polyethoxylated sorbitan ester” refers to a compound, or mixture thereof, derived from the ethoxylation of a sorbitan ester. The polyoxethylene portion of the compound can be between the fatty ester and the sorbitan moiety. As used herein, the term “sorbitan ester” refers to a compound, or mixture of compounds, derived from the esterification of sorbitol and at least one fatty acid. Fatty acids useful for deriving the polyethoyxlated sorbitan esters include, but are not limited to, those described herein. In some embodiments, the polyoxyethylene portion of the compound or mixture has about 2 to about 200 oxyethylene units. In some embodiments, the polyoxyethylene portion of the compound or mixture has about 2 to about 100 oxyethylene units. In some embodiments, the polyoxyethylene portion of the compound or mixture has about 4 to about 80 oxyethylene units. In some embodiments, the polyoxyethylene portion of the compound or mixture has about 4 to about 40 oxyethylene units. In some embodiments, the polyoxyethylene portion of the compound or mixture has about 4 to about 20 oxyethylene units. Suitable polyethoxylated sorbitan esters include, but are not limited to the Tween™ series (available from Uniqema), which includes Tween 20 (POE (20) sorbitan monolaurate), 21 (POE (4) sorbitan monolaurate), 40 (POE (20) sorbitan monopalmitate), 60 (POE (20) sorbitan monostearate), 60K (POE (20) sorbitan monostearate), 61 (POE (4) sorbitan monostearate), 65 (POE (20) sorbitan tristearate), 80 (POE (20) sorbitan monooleate), 80K (POE (20) sorbitan monooleate), 81 (POE (5) sorbitan monooleate), and 85 (POE (20) sorbitan trioleate). As used herein, the abbreviation “POE” refers to polyoxyethylene. The number following the POE abbreviation refers to the number of oxyethylene repeat units in the compound. Other suitable polyethoxylated sorbitan esters include the polyoxyethylene sorbitan fatty acid esters listed in R. C. Rowe and P. J. Shesky, Handbook of pharmaceutical excipients, (2006), 5th ed., which is incorporated herein by reference in its entirety. In some embodiments, the polyethoxylated sorbitan ester is a polysorbate. In some embodiments, the polyethoxylated sorbitan ester is polysorbate 20.

As used herein, the term “glyceryl fatty esters” refers to mono-, di- or triglycerides of fatty acids. The glyceryl fatty esters may be optionally substituted with sulfonic acid groups, or pharmaceutically acceptable salts thereof. Suitable fatty acids for deriving glycerides of fatty acids include, but are not limited to, those described herein. In some embodiments, the glyceryl fatty ester is a mono-glyceride of a fatty acid having 12 to 18 carbon atoms. In some embodiments, the glyceryl fatty ester is glyceryl stearate.

As used herein, the term “triglycerides” refers to a triglyceride of a fatty acid. In some embodiments, the triglyceride is medium chain triglycerides.

As used herein, the term “alkylene glycol” refers to a group of formula-O-alkylene-, wherein the alkylene group has 2 to 6, 2 to 4, or 2 to 3 carbon atoms. In some embodiments, the alkylene glycol is propylene glycol (1,2-propanediol).

As used herein, the term “polyethylene glycol” refers to a polymer containing ethylene glycol monomer units of formula —O—CH₂—CH₂—. Suitable polyethylene glycols may have a free hydroxyl group at each end of the polymer molecule, or may have one or more hydroxyl groups etherified with a lower alkyl, e.g., a methyl group. Also suitable are derivatives of polyethylene glycols having esterifiable carboxy groups. Polyethylene glycols useful in the present invention can be polymers of any chain length or molecular weight, and can include branching. In some embodiments, the average molecular weight of the polyethylene glycol is from about 200 to about 9000. In some embodiments, the average molecular weight of the polyethylene glycol is from about 200 to about 5000. In some embodiments, the average molecular weight of the polyethylene glycol is from about 200 to about 900. In some embodiments, the average molecular weight of the polyethylene glycol is about 400. Suitable polyethylene glycols include, but are not limited to polyethylene glycol-200, polyethylene glycol-300, polyethylene glycol-400, polyethylene glycol-600, and polyethylene glycol-900. The number following the dash in the name refers to the average molecular weight of the polymer.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

### Methods

The topical skin formulations of the invention are useful in treating skin disorders. Provided herein is a method of treating a skin disorder in a patient in need thereof, comprising applying the topical skin formulation described herein to an area of skin of said patient.

In some embodiments, the skin disorder is atopic dermatitis. In some embodiments, the skin disorder is psoriasis. In some embodiments, the skin disorder is non-segmental vitiligo. In some embodiments, the skin disorder is skin sensitization, skin irritation, skin rash, contact dermatitis or allergic contact sensitization. In some embodiments, the skin disorder is a bullous skin disorder. In some embodiments, the bullous skin disorder is vulgaris (PV) or bullous pemphigoid (BP). In some embodiments, the skin disorder is hidradenitis suppurativa. In some embodiments, the skin disorder is lichen planus. In some embodiments, the skin disorder is lichen sclerosus. In some embodiments, the skin disorder is alopecia. In some embodiments, the skin disorder is alopecia areata. In some embodiments, the skin disorder is frontal fibrosing alopecia. In some embodiments, the skin disorder lichen planopilaris. In some embodiments, the skin disorder seborrheic dermatitis.

Provided herein is a topical skin formulation for use in treatment of a skin disorder in a patient in need thereof.

Provided herein is use of a topical skin formulation for the preparation of a medicament for use in treatment of a skin disorder in a patient in need thereof.

In some embodiments, the skin disorder is an autoimmune bullous skin disorder such as pemphigus vulgaris (PV) or bullous pemphigoid (BP). In some embodiments, the skin disorder is psoriasis (for example, psoriasis vulgaris), atopic dermatitis, skin rash, skin irritation, skin sensitization (e.g., contact dermatitis or allergic contact dermatitis). For example, certain substances including some pharmaceuticals when topically applied can cause skin sensitization. In some embodiments, co-administration or sequential administration of the topical formulations of the invention together with the agent causing unwanted sensitization can be helpful in treating such unwanted sensitization or dermatitis.

The present invention further provides a method of treating dermatological side effects of other pharmaceuticals by administration of the compound of the invention. For example, numerous pharmaceutical agents result in unwanted allergic reactions which can manifest as acneiform rash or related dermatitis. Example pharmaceutical agents that have such undesirable side effects include anti-cancer drugs such as gefitinib, cetuximab, erlotinib, and the like. The formulations of the invention can be administered systemically or topically (e.g., localized to the vicinity of the dermatitis) in combination with (e.g., simultaneously or sequentially) the pharmaceutical agent having the undesirable dermatological side effect. In some embodiments, the formulation of the invention can be administered topically together with one or more other pharmaceuticals, where the other pharmaceuticals when topically applied in the absence of a formulation of the invention cause contact dermatitis, allergic contact sensitization, or similar skin disorder. Accordingly, formulation of the invention include topical formulations further comprising an additional pharmaceutical agent which can cause dermatitis, skin disorders, or related side effects.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

As used herein, the term “treating” or “treatment” refers to one or more of (1) preventing the disease; for example, preventing a disease, condition or disorder in an individual who may be predisposed to the disease, condition or disorder but does not yet experience or display the pathology or symptomatology of the disease; (2) inhibiting the disease; for example, inhibiting a disease, condition or disorder in an individual who is experiencing or displaying the pathology or symptomatology of the disease, condition or disorder (i.e., arresting further development of the pathology and/or symptomatology); and (3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

### Combination Therapies

One or more additional pharmaceutical agents such as, for example, chemotherapeutics, anti-inflammatory agents, steroids, immunosuppressants, as well as Bcr-Abl, Flt-3, RAF and FAK kinase inhibitors such as, for example, those described in WO 2006/056399, or other agents can be used in combination with the formulations of the present invention for treatment of JAK-associated diseases, disorders or conditions. The one or more additional pharmaceutical agents can be administered to a patient simultaneously or sequentially.

Example chemotherapeutic include proteosome inhibitors (e.g., bortezomib), thalidomide, revlimid, and DNA-damaging agents such as melphalan, doxorubicin, cyclophosphamide, vincristine, etoposide, carmustine, and the like.

Example steroids include corticosteroids such as dexamethasone or prednisone. Example Bcr-Abl inhibitors include the compounds, and pharmaceutically acceptable salts thereof, of the genera and species disclosed in U.S. Pat. No. 5,521,184, WO 04/005281, and U.S. Ser. No. 60/578,491.

Example suitable Flt-3 inhibitors include compounds, and their pharmaceutically acceptable salts, as disclosed in WO 03/037347, WO 03/099771, and WO 04/046120.

Example suitable RAF inhibitors include compounds, and their pharmaceutically acceptable salts, as disclosed in WO 00/09495 and WO 05/028444.

Example suitable FAK inhibitors include compounds, and their pharmaceutically acceptable salts, as disclosed in WO 04/080980, WO 04/056786, WO 03/024967, WO 01/064655, WO 00/053595, and WO 01/014402.

In some embodiments, the formulations of the invention can be used in combination with one or more other kinase inhibitors including imatinib, particularly for treating patients resistant to imatinib or other kinase inhibitors.

In some embodiments, a corticosteroid such as dexamethasone is administered to a patient in combination with the compound of the invention where the dexamethasone is administered intermittently as opposed to continuously.

### Labeled Compounds and Assay Methods

Another aspect of the present invention relates to formulations comprising a labeled active compound (radio-labeled, fluorescent-labeled, etc.) that would be useful not only in imaging techniques but also in assays, both in vitro and in vivo, for localizing and quantitating JAK in tissue samples, including human, and for identifying JAK ligands by inhibition binding of a labeled compound. Accordingly, the present invention includes JAK assays that contain such labeled compounds.

The present invention further includes formulations of an isotopically-labeled compound. An “isotopically” or “radio-labeled” compound is a compound where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro JAK labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled” or “labeled compound” is a compound that has incorporated at least one radionuclide. In some embodiments the radionuclide is selected from the group consisting of ³H, ¹⁴C, ¹²⁵I, ³⁵S and ⁸²Br.

### Kits

The present invention also includes pharmaceutical kits useful, for example, in the treatment or prevention of JAK-associated diseases or disorders, such as cancer, which include one or more containers containing a topical skin formulation of the invention. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### EMBODIMENTS

1. A topical skin formulation, wherein the topical formulation is an oil-in-water emulsion, comprising:
about 0.5% to about 1.5% of a pharmaceutically acceptable salt of ruxolitinib on a free base basis by weight of the formulation;
an aqueous component;
an oil component
an non-ionic emulsifier component; and
a pharmaceutically acceptable acid.

2. The topical skin formulation of embodiment 1, wherein the pharmaceutically acceptable acid is selected from phosphoric acid, citric acid, and lactic acid.

3. The topical skin formulation of embodiment 1, wherein the pharmaceutically acceptable acid is phosphoric acid.

4. The topical skin formulation according to embodiment 3, wherein the phosphoric acid is present in an amount of about 0.1% to about 0.5% by weight of the formulation.

5. The topical skin formulation according to embodiment 3, wherein the phosphoric acid is present in an amount of about 0.1% to about 0.3% by weight of the formulation.

6. The topical skin formulation according to embodiment 3, wherein the phosphoric acid is present in an amount of about 0.15% to about 0.25% by weight of the formulation.

7. The topical skin formulation according to embodiment 3, wherein the phosphoric acid is present in an amount of about 0.2% by weight of the formulation.

8. The topical skin formulation of embodiment 1, wherein the pharmaceutically acceptable acid is citric acid or lactic acid.

9. The topical skin formulation according to embodiment 8, wherein the citric acid or lactic acid is present in an amount of about 1% to about 5% by weight of the formulation.

10. The topical skin formulation according to embodiment 8, wherein the citric acid or lactic acid is present in an amount of about 2% to about 5% by weight of the formulation.

11. The topical skin formulation according to embodiment 8, wherein the citric acid or lactic acid is present in an amount of about 3% to about 5% by weight of the formulation.

12. The topical skin formulation according to any one of embodiments 1-11, wherein the oil component comprises one or more substances independently selected from petrolatums, fatty alcohols, mineral oils, triglycerides, and silicone oils.

13. The topical skin formulation according to any one of embodiments 1-11, wherein the oil component comprises one or more substances independently selected from white petrolatum, cetyl alcohol, stearyl alcohol, light mineral oil, medium chain triglycerides, and dimethicone 350.

14. The topical skin formulation according to any one of embodiments 1-11, wherein the oil component comprises one or more petrolatums, one or more fatty alcohols, one or more mineral oils, one or more triglycerides, and one or more silicone oils.

15. The topical skin formulation according to any one of embodiments 1-11, wherein the oil component comprises white petrolatum, cetyl alcohol, stearyl alcohol, light mineral oil, medium chain triglycerides, and dimethicone 350.

16. The topical skin formulation according to any one of embodiments 1-11, wherein the oil component comprises, by weight of the formulation:
about 3% to about 5% of one or more mineral oils;
about 6% to about 8% of one or more petrolatums;
about 4% to about 6% of one or more fatty alcohols;
about 0.5% to about 1.5% of one of more silicone oils; and
about 4% to about 6% of one or more trigylcerides.

17. The topical skin formulation according to any one of embodiments 1-11, wherein
the oil component comprises, by weight of the formulation:
about 3% to about 5% of light mineral oil;
about 6% to about 8% of white petrolatum;
about 4% to about 6% of a mixture of cetyl alcohol and stearyl alcohol;
about 0.5% to about 1.5% of dimethicone 350; and
about 4% to about 6% of medium chain trigylcerides.

18. The topical skin formulation according to any one of embodiments 1-11, wherein
the oil component comprises, by weight of the formulation:
about 4% of light mineral oil;
about 7% of white petrolatum;
about 3% of cetyl alcohol;
about 1.75% of stearyl alcohol;
about 1% of dimethicone 350; and
about 5% of medium chain trigylcerides.

19. The topical skin formulation according to any one of embodiments 1-18, wherein the non-ionic non-ionic emulsifier component comprises one or more substances independently selected from glyceryl fatty esters and sorbitan fatty esters.

20. The topical skin formulation according to any one of embodiments 1-18, wherein the non-ionic emulsifier component comprises one or more substances independently selected from glyceryl stearate, and polysorbate 20.

21. The topical skin formulation according to any one of embodiments 1-18, wherein the non-ionic non-ionic emulsifier component comprises one or more glyceryl fatty esters and one or more sorbitan fatty esters.

22. The topical skin formulation according to any one of embodiments 1-18, wherein the non-ionic emulsifier component comprises glyceryl stearate, and polysorbate 20.

23. The topical skin formulation according to any one of embodiments 1-22, wherein the non-ionic emulsifier component is present in an amount of about 2% to about 6% by weight of the formulation.

24. The topical skin formulation according to any one of embodiments 1-22, wherein the non-ionic emulsifier component is present in an amount of about 3% to about 5% by weight of the formulation.

25. The topical skin formulation according to any one of embodiments 1-22, wherein the non-ionic emulsifier component is present in an amount of about 4% to about 5% by weight of the formulation.

26. The topical skin formulation according to any one of embodiments 1-18, wherein the non-ionic emulsifier component is present in an amount of about 2.5% to about 3.5% of glyceryl stearate by weight of the formulation and about 1% to about 1.5% % of polysorbate 20 by weight of the formulation.

27. The topical skin formulation according to any one of embodiments 1-18, wherein the non-ionic emulsifier component is present in an amount of about 3% of glyceryl stearate by weight of the formulation and about 1.25% of polysorbate 20 by weight of the formulation.

28. The topical skin formulation according to any one of embodiments 1-27, wherein the oil component and the non-ionic emulsifier component are combined into an oil phase.

29. The topical skin formulation according to any one of embodiments 1-28, wherein the aqueous component comprises water and one or more substances independently selected from C₁₋₃ alkyl parabens, alkylene glycols, polyalkylene glycols; polysaccharides, and chelating agents.

30. The topical skin formulation according to any one of embodiments 1-28, wherein the aqueous component comprises water, one or more C₁₋₃ alkyl parabens, one or more alkylene glycols, one or more polyalkylene glycols; one or more polysaccharides, and one or more chelating agents.

31. The topical skin formulation according to any one of embodiments 1-28, wherein the aqueous component comprises water and one or more substances independently selected from methyl paraben, propyl paraben, propylene glycol, polyethylene glycol 200, xanthan gum, and edetate disodium.

32. The topical skin formulation according to any one of embodiments 1-28, wherein the aqueous component comprises water, methyl paraben, propyl paraben, propylene glycol, polyethylene glycol 200, xanthan gum, and edetate disodium.

33. The topical skin formulation according to any one of embodiments 1-28, wherein
the aqueous component comprises, by weight of the formulation:
about 40% to about 60% water;
about 10 to about 20% of one or more alkylene glycols;
about 5% to about 10% of one or more polyalkylene glycols;
about 0.03% to about 0.2% of one or more C₁₋₃ alkyl parabens;
about 0.3% to about 0.5% of one or more polysaccharides; and
about 0.01% to about 0.1% of one or more chelating agents.

34. The topical skin formulation according to any one of embodiments 1-28, wherein
the aqueous component comprises, by weight of the formulation:
about 45% to about 50% water;
about 13 to about 17% of one or more alkylene glycols;
about 6% to about 8% of one or more polyalkyelene glycols;
about 0.05% to about 0.2% of one or more C₁₋₃ alkyl parabens;
about 0.3% to about 0.5% of one or more polysaccharides; and about 0.03% to about 0.07% of one or more chelating agents.

35. The topical skin formulation according to any one of embodiments 1-28, wherein
the aqueous component comprises, by weight of the formulation:
about 45% to about 50% water;
about 14 to about 16% of one or more alkylene glycols;
about 6% to about 8% of one or more polyalkyelene glycols;
about 0.05% to about 0.2% of one or more C₁₋₃ alkyl parabens;
about 0.3% to about 0.5% of one or more polysaccharides; and
about 0.04% to about 0.06% of one or more chelating agents.

36. The topical skin formulation according to any one of embodiments 1-28, wherein
the aqueous component comprises, by weight of the formulation:
about 45% to about 50% water;
about 13 to about 17% of propylene glycol;
about 6% to about 8% of polyethylene glycol 200;
about 0.05% to about 0.15% of methyl paraben;
about 0.01% to about 0.1% of propyl paraben;
about 0.3% to about 0.5% of xanthan gum;
about 0.03% to about 0.07% of edetate disodium.

37. The topical skin formulation according to any one of embodiments 1-28, wherein
the aqueous component comprises, by weight of the formulation:
about 45% to about 50% water;
about 14 to about 16% of propylene glycol;
about 6% to about 8% of polyethylene glycol 200;
about 0.08% to about 0.12% of methyl paraben;
about 0.03% to about 0.08% of propyl paraben;
about 0.3% to about 0.5% of xanthan gum; and
about 0.04% to about 0.06% of edetate disodium.

38. The topical skin formulation according to any one of embodiments 1-28, wherein
the aqueous component comprises, by weight of the formulation:
about 45% to about 50% water;
about 15% of propylene glycol;
about 7% of polyethylene glycol 200;
about 0.1% of methyl paraben;
about 0.05% of propyl paraben;
about 0.4% of xanthan gum; and
about 0.05% of edetate disodium.

39. The topical skin formulation according to any one of embodiments 1-11, wherein the oil component comprises from about 10% to about 30% by weight of the formulation.

40. The topical skin formulation according to any one of embodiments 1-11, wherein the oil component comprises from about 15% to about 30% by weight of the formulation.

41. The topical skin formulation according to any one of embodiments 1-11, wherein the oil component comprises from about 20% to about 27% by weight of the formulation.

42. The topical skin formulation according to any one of embodiments 1-11, wherein oil component comprises from about 20% to about 25% by weight of the formulation.

43. The topical skin formulation according to any one of embodiments 39-42, wherein the oil component comprises one or more substances independently selected from petrolatums, fatty alcohols, mineral oils, triglycerides, and silicone oils.

44. The topical skin formulation according to any one of embodiments 39-42, wherein the oil component comprises one or more substances independently selected from white petrolatum, cetyl alcohol, stearyl alcohol, light mineral oil, medium chain triglycerides, and dimethicone 350.

45. The topical skin formulation according to any one of embodiments 39-42, wherein the oil component comprises one or more petrolatums, one or more fatty alcohols, one or more mineral oils, one or more triglycerides, and one or more silicone oils.

46. The topical skin formulation according to any one of embodiments 39-42, wherein the oil component comprises white petrolatum, cetyl alcohol, stearyl alcohol, light mineral oil, medium chain triglycerides, and dimethicone 350.

47. The topical skin formulation according to any one of embodiments 1-11 and 39-42, wherein the aqueous component comprises from about 40% to about 90% by weight of the formulation.

48. The topical skin formulation according to any one of embodiments 1-11 and 39-42, wherein the aqueous component comprises from about 50% to about 80% by weight of the formulation.

49. The topical skin formulation according to any one of embodiments 1-11 and 39-42, wherein the aqueous component comprises from about 60% to about 80% by weight of the formulation.

50. The topical skin formulation according to any one of embodiments 47-49, wherein the aqueous component comprises water and one or more substances independently selected from C₁₋₃ alkyl parabens, alkylene glycols, polyalkylene glycols; polysaccharides, and chelating agents.

51. The topical skin formulation according to any one of embodiments 47-49, wherein the aqueous component comprises water, one or more C₁₋₃ alkyl parabens, one or more alkylene glycols, one or more polyalkylene glycols; one or more polysaccharides, and one or more chelating agents.

52. The topical skin formulation according to any one of embodiments 47-49, wherein the aqueous component comprises water and one or more substances independently selected from methyl paraben, propyl paraben, propylene glycol, polyethylene glycol 200, xanthan gum, and edetate disodium.

53. The topical skin formulation according to any one of embodiments 47-49, wherein the aqueous component comprises water, methyl paraben, propyl paraben, propylene glycol, polyethylene glycol 200, xanthan gum, and edetate disodium.

54. The topical skin formulation according to any one of embodiments 47-49, wherein the water comprises from about 40% to about 60% of water by weight of the formulation.

55. The topical skin formulation according to any one of embodiments 47-49, wherein the water comprises from about 45% to about 55% of water by weight of the formulation.

56. The topical skin formulation according to any one of embodiments 1-11 and 39-55, the non-ionic emulsifier component is present in an amount of about 2% to about 6% by weight of the formulation.

57. The topical skin formulation according to any one of embodiments 1-11 and 39-55, the non-ionic emulsifier component is present in an amount of about 3% to about 5% by weight of the formulation.

58. The topical skin formulation according to any one of embodiments 1-11 and 39-55, the non-ionic emulsifier component is present in an amount of about 4% to about 5% by weight of the formulation.

59. The topical skin formulation according to any one of embodiments 56-58, wherein the non-ionic non-ionic emulsifier component comprises one or more substances independently selected from glyceryl fatty esters and sorbitan fatty esters.

60. The topical skin formulation according to any one of embodiments 56-58, wherein the non-ionic emulsifier component comprises one or more substances independently selected from glyceryl stearate, and polysorbate 20.

61. The topical skin formulation according to any one of embodiments 56-58, wherein the non-ionic non-ionic emulsifier component comprises one or more glyceryl fatty esters and one or more sorbitan fatty esters.

62. The topical skin formulation according to any one of embodiments 56-58, wherein the non-ionic emulsifier component comprises glyceryl stearate, and polysorbate 20.

63. The topical skin formulation according to any one of embodiments 1-62, wherein the pharmaceutically acceptable salt of ruxolitinib is ruxolitinib phosphate, ruxolitinib chloride, ruxolitinib mesylate, ruxolitinib maleate, ruxolitinib sulfate, ruxolitinib ediyslate, ruxolitinib napadisylate, ruxolitinib acesulfmate, ruxolitinib oxalate, ruxolitinib L-tartrate, ruxolitinib hydrobromide, and ruxolitinib hydroiodate.

64. The topical skin formulation according to any one of embodiments 1-62, wherein the pharmaceutically acceptable salt of ruxolitinib is ruxolitinib phosphate.

65. The topical skin formulation according to any one of embodiments 1-62, wherein the pharmaceutically acceptable salt of ruxolitinib is ruxolitinib chloride.

66. The topical skin formulation according to any one of embodiments 1-62, wherein the pharmaceutically acceptable salt of ruxolitinib is ruxolitinib mesylate.

67. The topical skin formulation according to any one of embodiments 1-62, wherein the pharmaceutically acceptable salt of ruxolitinib is ruxolitinib maleate.

68. The topical skin formulation according to any one of embodiments 1-62, wherein the pharmaceutically acceptable salt of ruxolitinib is ruxolitinib sulfate.

69. The topical skin formulation according to any one of embodiments 1-68, wherein the pharmaceutically acceptable salt of ruxolitinib is present in an amount of about 0.5% by weight of the formulation on a free base basis.

70. The topical skin formulation according to any one of embodiments 1-68, wherein the pharmaceutically acceptable salt of ruxolitinib is present in an amount of about 0.75% by weight of the formulation on a free base basis.

71. The topical skin formulation according to any one of embodiments 1-68, wherein the pharmaceutically acceptable salt of ruxolitinib is present in an amount of about 1% by weight of the formulation on a free base basis.

72. The topical skin formulation according to any one of embodiments 1-68, wherein the pharmaceutically acceptable salt of ruxolitinib is present in an amount of about 1.5% by weight of the formulation on a free base basis.

73. The topical skin formulation according to any one of embodiments 1-72, further comprising about 0.1% to about 0.8% of phenoxyethanol.

74. The topical skin formulation according to any one of embodiments 1-72, further comprising about 0.4% to about 0.6% of phenoxyethanol.

75. The topical skin formulation according to any one of embodiments 1-72, further comprising about 0.5% of phenoxyethanol.

76. The topical skin formulation according to embodiment 1, wherein the topical skin formulation comprises (by weight of the formulation):
about 10% to about 30% of the oil component;
about 40% to about 90% of the aqueous component;
about 0.5% to about 1.5% of the pharmaceutically acceptable salt of ruxolitinib; and
about 0.1% to about 0.5% of phosphoric acid.

77. The topical skin formulation according to embodiment 1, wherein the topical skin formulation comprises (by weight of the formulation):
about 15% to about 30% of the oil component;
about 50% to about 80% of the aqueous component;
about 0.5% to about 1.5% of the pharmaceutically acceptable salt of ruxolitinib; and
about 0.1% to about 0.5% of phosphoric acid.

78. The topical skin formulation according to embodiment 1, wherein the topical skin formulation comprises (by weight of the formulation):
about 20% to about 27% of the oil component;
about 60% to about 80% of the aqueous component;
about 0.5% to about 1.5% of the pharmaceutically acceptable salt of ruxolitinib; and
about 0.1% to about 0.5% of phosphoric acid.

79. The topical skin formulation according to any one of embodiments 76-78, wherein the aqueous component comprises about 40% to about 60% of water by weight of the formulation.

80. The topical skin formulation according to any one of embodiments 76-78, wherein the aqueous component comprises about 45% to about 50% of water by weight of the formulation.

81. The topical skin formulation according to any one of embodiments 1-80, wherein the pH of the formulation is about 2.6 to about 3.3.

82. The topical skin formulation according to any one of embodiments 1-81, wherein the formulation is a cream.

83. The topical skin formulation according to any one of embodiments 1-81, wherein the formulation is a solubilized cream.

84. A method of treating a skin disorder in a patient in need thereof, comprising applying the topical skin formulation according to any one of embodiments 1-83 to an area of skin of said patient.

85. The method of embodiment 84, wherein the skin disorder is atopic dermatitis.

86. The method of embodiment 84, wherein the skin disorder is psoriasis.

87. The method of embodiment 84, wherein the skin disorder is non-segmental vitiligo.

88. The method of embodiment 84, wherein the skin disorder is skin sensitization, skin irritation, skin rash, contact dermatitis or allergic contact sensitization.

89. The method of embodiment 84, wherein the skin disorder is a bullous skin disorder.

90. The method of embodiment 84, wherein the bullous skin disorder is vulgaris (PV) or bullous pemphigoid (BP).

91. The method of embodiment 84, wherein the skin disorder is hidradenitis suppurativa.

92. The method of embodiment 84, wherein the skin disorder is lichen planus.

93. The method of embodiment 84, wherein the skin disorder is lichen sclerosus.

94. The method of embodiment 84, wherein the skin disorder is alopecia.

95. The method of embodiment 84, wherein the skin disorder is alopecia areata.

96. The topical skin formulation according to any one of embodiments 1-83 for use in treatment of a skin disorder in a patient in need thereof.

97. Use of the topical skin formulation according to any one of embodiments 1-83 for the preparation of a medicament for use in treatment of a skin disorder in a patient in need thereof.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. In some embodiments, the present invention provides topical skin formulations comprising the components specified in the example formulations (e.g., Example 3), wherein the components are present in about the amounts in Tables 2-5.

### EXAMPLES

### Example 1: Ruxolitinib ((3R)-3-Cyclopentyl-3-[4-(7H-pyrrolo[2,3-d]pyrimidin-4-yl)-1H-pyrazol-1-yl]propanenitrile) and (3S)-3-Cyclopentyl-3-[4-(7H-pyrrolo[2,3-d]pyrimidin-4-yl)-1H-pyrazol-1-yl]propanenitrile

### Step 1. (2E)- and (2Z)-3-Cyclopentylacrylonitrile

To a solution of 1.0 M potassium tert-butoxide in THF (235 mL) at 0° C. was added dropwise a solution of diethyl cyanomethylphosphonate (39.9 mL, 0.246 mol) in THF (300 mL). The cold bath was removed and the reaction was warmed to room temperature followed by recooling to 0° C., at which time a solution of cyclopentanecarbaldehyde (22.0 g, 0.224 mol) in THF (60 mL) was added dropwise. The bath was removed and the reaction warmed to ambient temperature and stirred for 64 hours. The mixture was partitioned between diethyl ether and water, the aqueous was extracted with three portions of ether, followed by two portions of ethyl acetate. The combined extracts were washed with brine, then dried over sodium sulfate, filtered and concentrated in vacuo to afford a mixture containing 24.4 g of olefin isomers which was used without further purification (89%).

¹H NMR (400 MHZ, CDCl₃): δ 6.69 (dd, 1H, trans olefin), 6.37 (t, 1H, cis olefin), 5.29 (dd, 1H, trans olefin), 5.20 (d, 1H, cis olefin), 3.07-2.95 (m, 1H, cis product), 2.64-2.52 (m, 1H, trans product), 1.98-1.26 (m, 16H).

### Step 2. (3R)- and (3S)-3-Cyclopentyl-3-[4-(7-[2-(trimethylsilyl) ethoxy]methyl-7H-pyrrolo[2,3-d]pyrimidin-4-yl)-1H-pyrazol-1-yl]propanenitrile

To a solution of 4-(1H-pyrazol-4-yl)-7-[2-(trimethylsilyl) ethoxy]methyl-7H-pyrrolo[2,3-d]pyrimidine (15.0 g, 0.0476 mol) in ACN (300 mL) was added 3-cyclopentylacrylonitrile (15 g, 0.12 mol) (as a mixture of cis and trans isomers), followed by DBU (15 mL, 0.10 mol). The resulting mixture was stirred at room temperature overnight. The ACN was evaporated. The mixture was diluted with ethyl acetate, and the solution was washed with 1.0 N HCl. The aqueous layer was back-extracted with three portions of ethyl acetate. The combined organic extracts were washed with brine, dried over sodium sulfate, filtered and concentrated. The crude product was purified by silica gel chromatography (gradient of ethyl acetate/hexanes) to yield a viscous clear syrup, which was dissolved in ethanol and evaporated several times to remove ethyl acetate, to afford 19.4 g of racemic adduct (93%). The enantiomers were separated by preparative-HPLC, (OD-H, 15% ethanol/hexanes) and used separately in the next step to generate their corresponding final product. The final products (see Step 3) stemming from each of the separated enantiomers were found to be active JAK inhibitors; however, the final product stemming from the second peak to elute from the preparative-HPLC was more active than its enantiomer.

¹H NMR (300 MHz, CDCl₃): δ 8.85 (s, 1H), 8.32 (s, 2H), 7.39 (d, 1H), 6.80 (d, 1H), 5.68 (s, 2H), 4.26 (dt, 1H), 3.54 (t, 2H), 3.14 (dd, 1H), 2.95 (dd, 1H), 2.67-2.50 (m, 1H), 2.03-1.88 (m, 1H), 1.80-1.15 (m, 7H), 0.92 (t, 2H), −0.06 (s, 9H); MS(ES): 437 (M+1).

### Step 3. (3R)- and (3S)-3-Cyclopentyl-3-[4-(7H-pyrrolo[2,3-d]pyrimidin-4-yl)-1H-pyrazol-1-yl]propanenitrile

To a solution of 3-cyclopentyl-3-[4-(7-[2-(trimethylsilyl) ethoxy]methyl-7H-pyrrolo[2,3-d]pyrimidin-4-yl)-1H-pyrazol-1-yl]propanenitrile (6.5 g, 0.015 mol, R or S enantiomer as isolated above) in DCM (40 mL) was added TFA (16 mL) and this was stirred for 6 hours. The solvent and TFA were removed in vacuo. The residue was dissolved in DCM and concentrated using a rotary evaporator two further times to remove as much as possible of the TFA. Following this, the residue was stirred with ethylenediamine (4 mL, 0.06 mol) in methanol (30 mL) overnight. The solvent was removed in vacuo, water was added and the product was extracted into three portions of ethyl acetate. The combined extracts were washed with brine, dried over sodium sulfate, decanted and concentrated to afford the crude product which was purified by flash column chromatography (eluting with a gradient of methanol/DCM). The resulting mixture was further purified by preparative-HPLC/MS (C18 eluting with a gradient of ACN/H₂O containing 0.15% NH₄OH) to afford product (2.68 g, 58%).

¹H NMR (400 MHz, D₆-dmso): δ 12.11 (br s, 1H), 8.80 (s, 1H), 8.67 (s, 1H), 8.37 (s, 1H), 7.60 (d, 1H), 6.98 (d, 1H), 4.53 (dt, 1H), 3.27 (dd, 1H), 3.19 (dd, 1H), 2.48-2.36 (m, 1H), 1.86-1.76 (m, 1H), 1.68-1.13 (m, 7H); MS(ES): 307 (M+1).

### Example 2: (R)-3-(4-(7H-pyrrolo[2,3-d]pyrimidin-4-yl)-1H-pyrazol-1-yl)-3-cyclopentylpropanenitrile (ruxolitinib) phosphoric acid salt

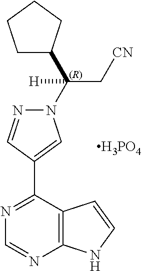

To a test tube was added (R)-3-(4-(7H-pyrrolo[2,3-d]pyrimidin-4-yl)-1H-pyrazol-1-yl)-3-cyclopentylpropanenitrile (153.5 mg) and phosphoric acid (56.6 mg) followed by isopropyl alcohol (IPA) (5.75 mL). The resulting mixture was heated to clear, cooled to room temperature, and then stirred for another 2 hours. The precipitate was collected by filtration and the cake was washed with 0.6 mL of cold IPA. The cake was dried under vacuum to constant weight to provide the final salt product (171.7 mg).

The phosphoric acid salt was shown to be a 1:1 salt by 1H NMR and crystallinity was confirmed by X-ray powder diffraction (XRPD). Differential scanning calorimetry (DSC) gave a sharp melting peak at about 198.66° C. The product showed little weight loss up to 200° C. by TGA.

### Example 3: Preparation of a Topical Skin Formulation

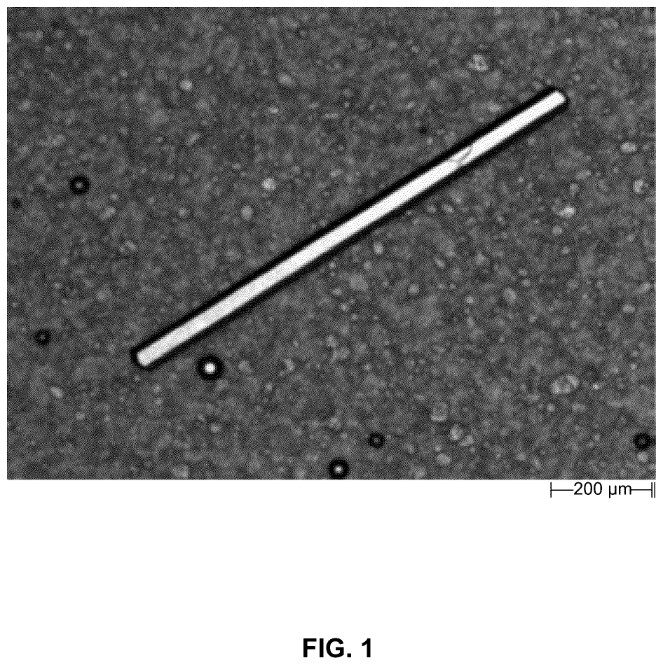

The presence of ruxolitinib dihydrate crystals has been observed in a small number of batches of ruxolitinib cream 1.5% (w/w) as prepared analogous to the formulation in Table 5 of U.S. Pat. No. 10,758,543, which is incorporated herein by reference in its entirety, but prepared at large scale (hereinafter, the “original formulation”). Ruxolitinib dihydrate can be characterized as summarized in US 20230399331, which is incorporated herein by reference in its entirety. When present in the cream, ruxolitinib dihydrate crystals are identified primarily by their distinct rod-shaped crystal habit (FIG. 1).

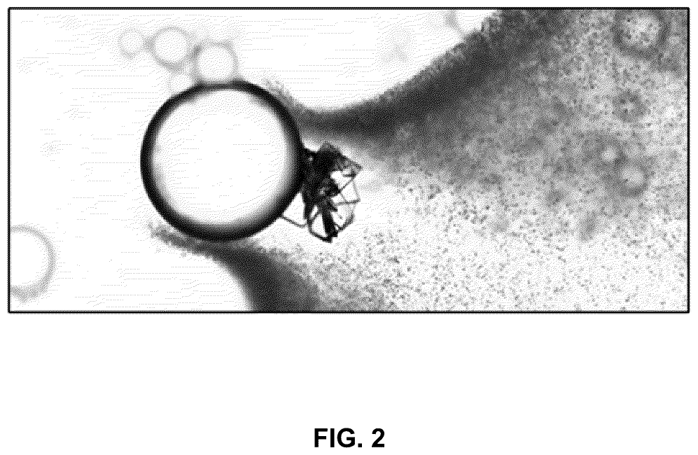

While not wishing to be bound by any theory, it is believed that the ruxolitinib dihydrate crystals results from dissociation of ruxolitinib phosphate in aqueous solution, followed by association of the free base molecules at the interface of dimethicone oil droplets (due to the low aqueous solubility of the free base and low interfacial tension with dimethicone drops inside oil droplet). This can potentially lead to disruption of the normally ordered structure of the oil droplet surface, resulting in the formation of silicone-in-oil droplets, and coalescence of regular smaller emulsion droplets into larger (amorphous) oil droplets. The free base can continue to accumulate at the droplet surface, until a critical crystallization concentration is reached, whereupon dihydrate crystallization can occur (see FIG. 2 showing the growth of dihydrate crystals at the oil droplet interface).

Unexpectedly, it has been found that the addition of an acid such as phosphoric or citric acid can stabilize the formulation against crystal formation. Without wishing to be bound by any particular theory, addition of the acid can drive the equilibrium dissociation of the ruxolitinib phosphate to the protonated species, thereby reducing the amount of the less soluble ruxolitinib free base in the formulation and at the emulsion interface, an in turn, improving the quality of the emulsion (e.g., the addition of the acid also helps stabilize the formulation by achieving a smaller and more uniform droplet size in the formulation).

Accordingly, a 900 kg batch of a topical skin formulation was prepared at a 900 kg scale as shown below in Table 1.

TABLE 1

| | Amount | | |
|---|---|---|---|
| | per Batch | % | |
| Component | (kg) | Weight | |
| Ruxolitinib Phosphate^{a} | 17.8 | 1.98 | (1.50^{b}) |
| Propylene Glycol | 135.0 | 15.00 | |
| Methylparaben | 0.90 | 0.10 | |
| Propylparaben | 0.45 | 0.05 | |
| Xanthan Gum | 3.60 | 0.40 | |
| Light Mineral Oil | 36.0 | 4.00 | |
| Glyceryl Stearate SE | 27.0 | 3.00 | |
| Polysorbate 20 | 11.25 | 1.25 | |
| White Petrolatum | 63.0 | 7.00 | |
| Cetyl Alcohol | 27.0 | 3.00 | |
| Stearyl Alcohol | 15.75 | 1.75 | |
| Dimethicone 350 | 9.0 | 1.00 | |
| Triglycerides, Medium Chain | 45.0 | 5.00 | |
| Purified Water | 438.2 | 48.69 | (48.72)^{c} |
| Phosphoric Acid 85% | 2.07 | 0.23 | (0.1955)^{c} |

| | | |
|---|---|---|
| Edetate Disodium | 0.45 | 0.05 |
| Polyethylene Glycol 200 | 63.0 | 7.00 |
| Phenoxyethanol | 4.50 | 0.50 |

^{a}Conversion factor for phosphate salt to free base is 0.758
^{b}Free base equivalent
^{c}Adjusted for the water in 85% phosphoric acid

The following procedure was used to prepare the batch.

### Step 1 Preparation of Oil Phase

Combine and warm to 68-72° C.,
light mineral oil
glyceryl stearate SE
polysorbate 20
white petrolatum
cetyl alcohol
stearyl alcohol
dimethicone 350
medium chain triglycerides

Maintain mixing until ready to use in Step 6.

### Step 2 Preparation of Presolution

Combine in order:
86.7% of the total propylene glycol
polyethylene glycol 200
edetate disodium
methylparaben
propylparaben
ruxolitinib phosphate

71.2% of the total purified water, preheated to 65-75° C.

Warm the mixture to 68-72° C. and hold at this temperature until ready to use in Step 4.

### Step 3 Preparation of Phosphoric Acid Solution

Combine:

The rest of the total purified water, preheated to 65-75° C. phosphoric acid (added as an 85% solution)

With a mixing speed of 100 rpm, warm the mixture to 68-72° C. Use this mixture in Step 4.

### Step 4 Dissolution of Ruxolitinib Phosphate

Combine in the main processing vessel:
Presolution from Step 2

Phosphoric acid solution from Step 3

Mix the aqueous phase for 180-240 minutes while maintaining the temperature at 68-72° C.

### Step 5 Xanthan Gum Hydration

Transfer:
xanthan gum
12.7% of the total propylene glycol

Mix the contents at a temperature of 68-72° C. This completes the Aqueous Phase.

### Step 6 Compounding (Emulsification)

Transfer:
the Oil Phase from Step 1
to the vessel, maintaining the temperature at 68-72° C. with recirculation.

### Step 7 First Cooling Step

Initiate cooling of the contents of the main processing vessel with a set point of 40° C. (38-42° C.).

### Step 8 Addition of Phenoxyethanol

At a product temperature of 38-42° C., add:
phenoxyethanol
the remaining propylene glycol
to the mixture and mix.

### Step 9 Final Cooling

Cooling the mixture with to a final product temperature 22-25° C.

The topical formulation was had a pH in the range of 2.6 to 3.3. The topical skin formulation of Example 3 was found to unexpectedly not contain any ruxolitinib dihydrate crystals over the preparation of multiple 900 kg batches, including through 18 months of stability testing at 25° C. and 60% relative humidity and 40° C. and 75% relative humidity.

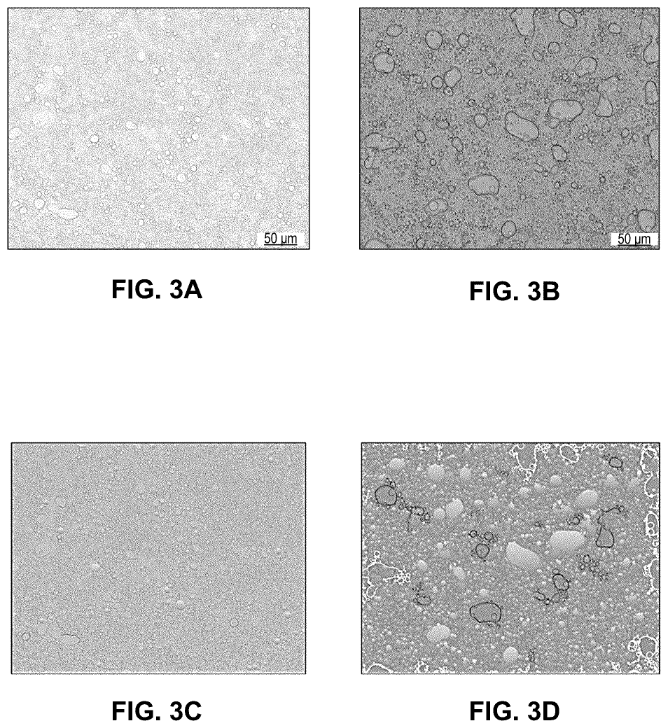

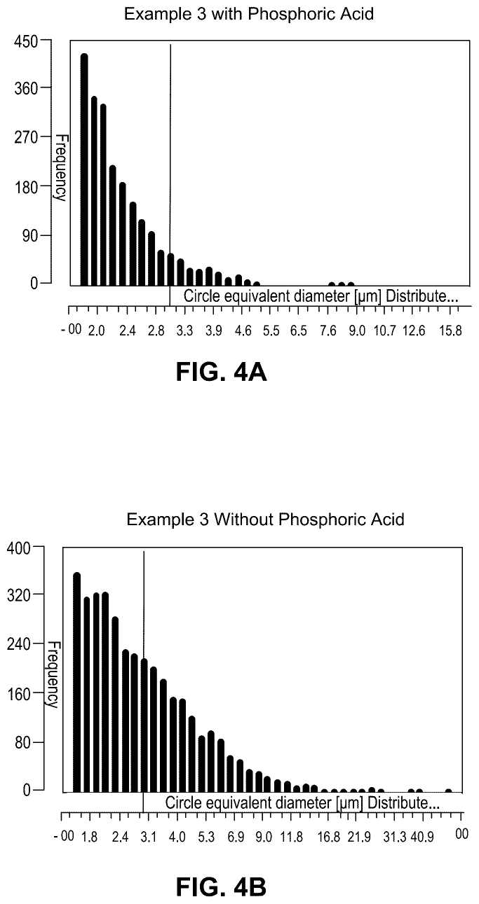

Automated image analysis obtained using Malvern Morphologi G3 presented in FIG. 3A-3D and FIG. 4A-4B-demonstrates the effect of phosphoric acid addition on reducing the droplet size and tightening the droplet size distribution in favor of a more uniform emulsion. FIG. 3A shows the raw microscopy image for the Example 3 formulation, while FIG. 3B shows the raw microscopy image for the original formulation without the phosphoric acid. FIG. 3C and FIG. 3D are the same microscopy images as in FIG. 3A and FIG. 3B, respectively, after the IMAGIC image analysis. FIG. 4A and FIG. 4B are the histograms showing the globule size distributions of the batches for the Example 3 formulation and the original formulation, respectively. The vertical line in the histogram plots corresponds to a 3.0 μm size cut-off. The histograms depict the dramatic decrease of globule size observed in the Example 3 formulation as compared to the original formulation.

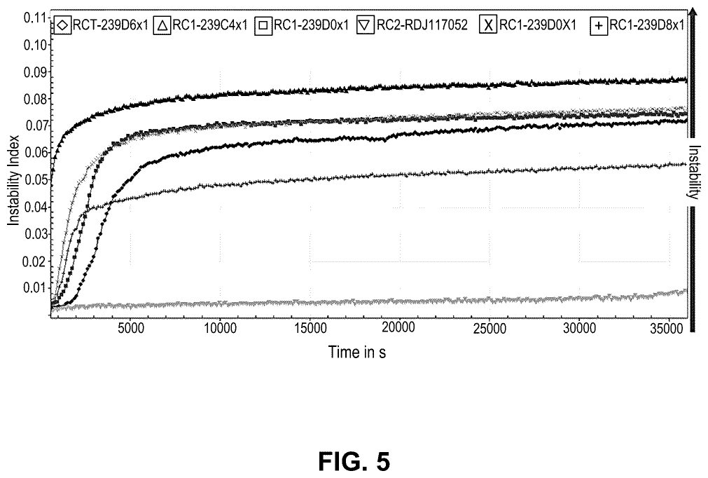

Further, demonstration of the improvement in the stability of the Example 3 formulation's emulsion brought about by inclusion of phosphoric acid is shown in FIG. 5. Using a LUMiSizer® Dispersion Analyzer, the stability of the original formulation was compared to a representative batch of the Example 3 formulation to rapidly assess product instability.

The LUMiSizer spins a sample of the cream in a cell under high g-force, rapidly simulating long term static stability hold, and measures the drift in particles throughout the height of the sample to determine any phase separation that may occur. Through computational evaluation of the data, it determines an ‘Instability Index’ of the product; the lower the Instability Index number the more stable the product.

The data shown in FIG. 5 shows the Example 3 formulation (bottom line, identified as RC2) to be far more stable (index number of 0.009) with no phase separation observed throughout the 10 hours runtime compared to the original formulations (all other lines, which are identified as RC1 and RCT)—the original formulation rapidly phase separating in the first hour of the test, leading to instability index numbers in the range of 0.056-0.087 at the end of the 10 hour test period. In FIG. 5, from the top of the graph to the bottom, the formulations in order are as follows: RC1-239C4X1, RC1-239D0X1 (two formulations), RC1-239D6X1, RC1-239D8X1, and RC2-RDJ117052.

Additionally, a second topical skin formulation batch was prepared by the process in Example 3 but having the composition as shown in Table 2. This was done to show the impact of lowering the ruxolitinib phosphate and phosphoric amounts in the formulation. 0.75% (w/w) ruxolitinib phosphate on a free base basis and approximately 0.1% phosphoric acid. The formulation was to be stable against crystal formulation through 18 months of stability testing at 25° C. and 60% relative humidity and 40° C. and 75% relative humidity.

TABLE 2

| | Amount per | % | |
|---|---|---|---|
| Component | Batch (kg) | Weight | |
| Ruxolitinib Phosphate^{a} | 8.91 | 0.99 | (0.75^{b}) |
| Propylene Glycol | 135 | 15.0 | |
| Methylparaben | 0.9 | 0.10 | |
| Propylparaben | 0.45 | 0.05 | |
| Xanthan Gum | 3.6 | 0.40 | |
| Light Mineral Oil | 36 | 4.00 | |
| Glyceryl Stearate SE | 27 | 3.00 | |
| Polysorbate 20 | 11.25 | 1.25 | |
| White Petrolatum | 63 | 7.00 | |
| Cetyl Alcohol | 27 | 3.00 | |
| Stearyl Alcohol | 15.75 | 1.75 | |
| Dimethicone 350 | 9 | 1.00 | |
| Triglycerides, Medium Chain | 45 | 5.00 | |
| Purified Water | 448.2 | 49.795 | |
| Phosphoric Acid 85% | 1.04 | 0.115 | (0.098)^{c} |

| | | |
|---|---|---|
| Edetate Disodium | 0.45 | 0.05 |
| Polyethylene Glycol 200 | 63 | 7.00 |
| Phenoxyethanol | 4.5 | 0.50 |

^{a}Conversion factor for phosphate salt to free base is 0.758
^{b}Free base equivalent
^{c}Adjusted for the water in 85% phosphoric acid

### Example 4. Seeding Experiments

In order to screen for the efficacy of various acids to stabilize the original formulation against ruxolitinib dihydrate formulation, screening experiments were undertaken where samples of the original formulation were seeded with ruxolitinib dihydrate crystals, followed by addition of an acid, followed by monitoring for crystal formation of the formulation via microscopy for seven days. The procedure below was utilized:
1. Five (5) g of 1.5% (w/w) ruxolitinib cream (on a free base basis) of the original formulation was seeded with approximately 2 mg (0.04-0.05 wt %) of the crystalline ruxolitinib dihydrate.
2. Approximately 0.1-2.0 equivalents of citric acid acid (to 1 equivalent of ruxolitinib dihydrate) or 0.05-5 equivalents of phosphoric acid (to 1 equivalent of ruxolitinib dihydrate) was added based on the equivalents of ruxolitinib dihydrate seed.
3. The samples were heated at 70° C. for one (1) hour with agitation.
4. The samples were gradually cooled to ambient temperature with agitation.
5. The samples were then monitored by microscopy for the presence of ruxolitinib dihydrate crystals.

Results of the seeding experiments with citric acid and phosphoric acid are shown below in Tables 3 and 4, respectively.

TABLE 3

| | | | | | | | |
|---|---|---|---|---|---|---|---|
| Ruxolitinib Dihydrate (mg) | 0 | 2.1 | 2.5 | 2.6 | 2.2 | 2.6 | 2.3 |
| Ruxolitinib Dihydrate (wt %) | 0 | 0.04 | 0.05 | 0.05 | 0.04 | 0.05 | 0.05 |
| Citric Acid (mg) | 0 | 0 | 5.2 | 10.0 | 25.1 | 52.1 | 100.1 |
| Citric Acid (equiv) | 0 | 0 | 0.1 | 0.2 | 0.5 | 1.0 | 2.0 |

| | Microscopy Results | | | | | | |
|---|---|---|---|---|---|---|---|
| Time (days) | (crystals observed?) | | | | | | |
| 0 | Yes | Yes | No | No | No | No | No |
| 4 | Yes | Yes | No | No | No | No | No |
| 7 | Yes | Yes | No | Yes | No | No | No |
| 11 | Yes | Yes | Yes | Yes | No | No | No |
| 14 | Yes | Yes | Yes | Yes | No | No | No |
| 21 | Yes | Yes | Yes | Yes | Yes | No | No |
| 31 | Yes | Yes | Yes | Yes | Yes | No | No |
| 35 | Yes | Yes | Yes | Yes | Yes | No | No |
| 39 | Yes | Yes | Yes | Yes | Yes | No | No |
| 47 | Yes | Yes | Yes | Yes | Yes | No | No |
| 53 | Yes | Yes | Yes | Yes | Yes | No | No |
| 61 | Yes | Yes | Yes | Yes | Yes | No | No |
| 81 | Yes | Yes | Yes | Yes | Yes | No | No |
| 102 | Yes | Yes | Yes | Yes | Yes | No | No |

TABLE 4

| | | | | | | | |
|---|---|---|---|---|---|---|---|
| Ruxolitinib Dihydrate (mg) | 0 | 2.3 | 2.3 | 2.5 | 2.6 | 2.5 | 2.6 |
| Ruxolitinib Dihydrate (wt %) | 0 | 0.05 | 0.05 | 0.05 | 0.05 | 0.05 | 0.05 |
| Phosphoric Acid (mg), 1N | 0 | 12 | 25.2 | 50.5 | 75 | 100 | 125 |
| aqueous solution | | | | | | | |
| Phosphoric Acid (equiv) | 0 | 0.05 | 0.1 | 0.2 | 0.3 | 0.4 | 0.5 |

| | Microscopy Results | | | | | | |
|---|---|---|---|---|---|---|---|
| Time (days) | (crystals observed?) | | | | | | |
| 0 | Yes | No | No | No | No | No | No |
| 5 | Yes | No | No | No | No | No | No |
| 11 | Yes | No | No | No | No | No | No |
| 14 | Yes | Yes | Yes | No | No | No | No |
| 20 | Yes | Yes | Yes | No | No | No | No |
| 24 | Yes | Yes | Yes | Yes | No | No | No |
| 28 | Yes | Yes | Yes | Yes | No | No | No |
| 36 | Yes | Yes | Yes | Yes | Yes | No | No |
| 42 | Yes | Yes | Yes | Yes | Yes | No | No |
| 50 | Yes | Yes | Yes | Yes | Yes | No | No |
| 70 | Yes | Yes | Yes | Yes | Yes | No | No |
| 91 | Yes | Yes | Yes | Yes | Yes | No | No |

Because no ruxolitinib dihydrate crystals were observed in Table 3 in spiked samples of ruxolitinib cream containing 1.0 equivalent and 2.0 equivalents of citric acid, the citric acid additive was further reduced to 0.7, 0.8, and 0.9 equivalents of citric acid, respectively, following the procedure detailed above. The results are shown in Table 5. However, the ruxolitinib dihydrate crystals were detected in these samples after less than 4 weeks, which means at least 1.0 equivalent of citric acid is needed to make the ruxolitinib cream acidic enough to prevent formation of the crystalline ruxolitinib dihydrate.

TABLE 5

| | | | | | |
|---|---|---|---|---|---|
| Ruxolitinib Dihydrate (mg) | 0 | 2 | 2 | 2 | 2 |
| Ruxolitinib Dihydrate (wt %) | 0 | 0.05 | 0.05 | 0.05 | 0.05 |
| Citric Acid (mg) | 0 | 0 | 35 | 40 | 45 |
| Citric Acid (equiv) | 0 | 0 | 0.7 | 0.8 | 0.9 |

| | |
|---|---|
| | Microscopy Results |
| Time (days) | (crystals observed?) |

| | | | | | |
|---|---|---|---|---|---|
| 0 | Yes | Yes | No | No | No |
| 4 | Yes | Yes | No | No | No |
| 12 | Yes | Yes | Yes | No | No |
| 18 | Yes | Yes | Yes | No | No |
| 26 | Yes | Yes | Yes | Yes | Yes |

Because no ruxolitinib dihydrate crystals were observed in Table 4 in spiked samples of ruxolitinib cream containing 0.4 equivalents and 0.5 equivalents of phosphoric acid after four months, at least 0.4 equivalents of phosphoric acid is needed to make the ruxolitinib cream acidic enough to prevent formation of the crystalline ruxolitinib dihydrate. When the added phosphoric acid was further reduced to 0.22, 0.25, and 0.28 equivalents, ruxolitinib dihydrate crystals were detected after less than 2 weeks (see Table 6)

TABLE 6

| | | | | | |
|---|---|---|---|---|---|
| Ruxolitinib Dihydrate (mg) | 0 | 2 | 2 | 2 | 2 |
| Ruxolitinib Dihydrate (wt %) | 0 | 0.05 | 0.05 | 0.05 | 0.05 |
| Phosphoric Acid (mg), 1N | 0 | 0 | 55.4 | 63 | 70.6 |
| aqueous solution | | | | | |
| Phosphoric Acid (equiv) | 0 | 0 | 0.22 | 0.25 | 0.28 |

| | |
|---|---|
| | Microscopy Results |
| Time (days) | (crystals observed?) |

| | | | | | |
|---|---|---|---|---|---|
| Yes | Yes | No | No | No | Yes |
| Yes | Yes | No | No | No | Yes |
| Yes | Yes | Yes | Yes | Yes | Yes |

### Example 5. Further Topical Skin Formulations

Additional formulations are prepared at a 900 kg scale according to the manufacturing procedure in Example 3, but using the amounts of excipients and API shown in Tables 7-9 below.

TABLE 7

| | Amount | | |
|---|---|---|---|
| | per Batch | % | |
| Component | (kg) | Weight | |
| Ruxolitinib Phosphate^{a} | 17.8 | 1.98 | (1.50^{b}) |
| Propylene Glycol | 135.0 | 15.00 | |
| Methylparaben | 0.90 | 0.10 | |
| Propylparaben | 0.45 | 0.05 | |
| Xanthan Gum | 3.60 | 0.40 | |
| Light Mineral Oil | 36.0 | 4.00 | |
| Glyceryl Stearate SE | 27.0 | 3.00 | |
| Polysorbate 20 | 11.25 | 1.25 | |
| White Petrolatum | 63.0 | 7.00 | |
| Cetyl Alcohol | 27.0 | 3.00 | |
| Stearyl Alcohol | 15.75 | 1.75 | |
| Dimethicone 350 | 9.0 | 1.00 | |
| Triglycerides, Medium Chain | 45.0 | 5.00 | |
| Purified Water | 438.2 | 48.69 | (48.78)^{c} |
| Phosphoric Acid 85% | 5.29 | 0.5882 | (0.5)^{c} |

| | | |
|---|---|---|
| Edetate Disodium | 0.45 | 0.05 |
| Polyethylene Glycol 200 | 63.0 | 7.00 |
| Phenoxyethanol | 4.50 | 0.50 |

^{a}Conversion factor for phosphate salt to free base is 0.758
^{b}Free base equivalent
^{c}Adjusted for the water in 85% phosphoric acid

TABLE 8

| | Amount | | |
|---|---|---|---|
| | per Batch | % | |
| Component | (kg) | Weight | |
| Ruxolitinib Phosphate^{a} | 17.8 | 1.98 | (1.50^{b}) |
| Propylene Glycol | 135.0 | 15.00 | |
| Methylparaben | 0.90 | 0.10 | |
| Propylparaben | 0.45 | 0.05 | |
| Xanthan Gum | 3.60 | 0.40 | |
| Light Mineral Oil | 36.0 | 4.00 | |
| Glyceryl Stearate SE | 27.0 | 3.00 | |
| Polysorbate 20 | 11.25 | 1.25 | |
| White Petrolatum | 63.0 | 7.00 | |
| Cetyl Alcohol | 27.0 | 3.00 | |
| Stearyl Alcohol | 15.75 | 1.75 | |
| Dimethicone 350 | 9.0 | 1.00 | |
| Triglycerides, Medium Chain | 45.0 | 5.00 | |
| Purified Water | 438.2 | 48.69 | (48.71)^{c} |
| Phosphoric Acid 85% | 1.06 | 0.1176 | (0.1)^{c} |

| | | |
|---|---|---|
| Edetate Disodium | 0.45 | 0.05 |
| Polyethylene Glycol 200 | 63.0 | 7.00 |
| Phenoxyethanol | 4.50 | 0.50 |

^{a}Conversion factor for phosphate salt to free base is 0.758
^{b}Free base equivalent
^{c}Adjusted for the water in 85% phosphoric acid

TABLE 9

| | Amount | | |
|---|---|---|---|
| | per Batch | % | |
| Component | (kg) | Weight | |
| Ruxolitinib Phosphate^{a} | 17.8 | 1.98 | (1.50^{b}) |
| Propylene Glycol | 135.0 | 15.00 | |
| Methylparaben | 0.90 | 0.10 | |
| Propylparaben | 0.45 | 0.05 | |
| Xanthan Gum | 3.60 | 0.40 | |
| Light Mineral Oil | 36.0 | 4.00 | |
| Glyceryl Stearate SE | 27.0 | 3.00 | |
| Polysorbate 20 | 11.25 | 1.25 | |
| White Petrolatum | 63.0 | 7.00 | |
| Cetyl Alcohol | 27.0 | 3.00 | |
| Stearyl Alcohol | 15.75 | 1.75 | |
| Dimethicone 350 | 9.0 | 1.00 | |
| Triglycerides, Medium Chain | 45.0 | 5.00 | |
| Purified Water | 438.2 | 48.69 | (48.74) |
| Phosphoric Acid 85% | 3.18 | 0.3529 | (0.3) ^{c} |

| | | |
|---|---|---|
| Edetate Disodium | 0.45 | 0.05 |
| Polyethylene Glycol 200 | 63.0 | 7.00 |
| Phenoxyethanol | 4.50 | 0.50 |

^{a}Conversion factor for phosphate salt to free base is 0.758
^{b}Free base equivalent
^{c} Adjusted for the water in 85% phosphoric acid

### Example 6. Additional Topical Skin Formulations

Additional formulations were prepared at a 1 kg scale according to the manufacturing procedure in Example 3 but using the amounts of excipients and API shown in Table 10 below.

TABLE 10

| | Compositions (% w/w) | | | | | | |
|---|---|---|---|---|---|---|---|
| | Originial | Variant | Variant | Variant | Variant | Variant | Variant |
| Component | formulation | 1* | 2* | 3 | 4 | 5* | 6* |
| Propylene | 15.00 | 15.00 | 15.00 | 15.00 | 15.00 | 16.50 | 16.50 |
| glycol | | | | | | | |
| Methyl paraben | 0.10 | 0.10 | 0.10 | 0.10 | 0.10 | 0.10 | 0.10 |
| Propyl paraben | 0.05 | 0.05 | 0.05 | 0.05 | 0.05 | 0.05 | 0.05 |
| Xanthan gum | 0.40 | 0.40 | 0.40 | 0.40 | 0.40 | 0.40 | 0.40 |
| Light mineral | 4.00 | 4.00 | 4.00 | 4.00 | 4.00 | 4.00 | 4.00 |
| oil | | | | | | | |
| Glyceryl | 3.00 | 3.00 | 3.00 | 3.00 | 3.00 | 3.00 | 3.00 |
| stearate | | | | | | | |
| Polysorbate 20 | 1.25 | 1.25 | 1.25 | 1.25 | 1.25 | 1.25 | 1.25 |
| White | 7.00 | 7.00 | 7.00 | 7.00 | 7.00 | 7.00 | 7.00 |
| petrolatum | | | | | | | |
| Cetyl alcohol | 3.00 | 3.00 | 3.00 | 3.00 | 3.00 | 3.00 | 3.00 |
| Stearyl alcohol | 1.75 | 1.75 | 1.75 | 1.75 | 1.75 | 1.75 | 1.75 |
| Dimethicone | 1.00 | 1.00 | 1.00 | 1.00 | 1.00 | 1.00 | 1.00 |
| 350 | | | | | | | |
| Medium chain | 5.00 | 5.00 | 5.00 | 5.00 | 5.00 | 5.00 | 5.00 |
| triglyceride | | | | | | | |
| Purified water | 48.92 | 48.42 | 47.92 | 46.92 | 44.92 | 46.72 | 42.72 |
| Edetate | 0.05 | 0.05 | 0.05 | 0.05 | 0.05 | 0.05 | 0.05 |
| disodium | | | | | | | |
| Polyethylene | 7.00 | 7.00 | 7.00 | 7.00 | 7.00 | 7.70 | 7.70 |
| glycol 200 | | | | | | | |
| Anhydrous | — | 0.50 | 1.00 | 2.00 | 4.00 | — | 4.00 |
| citric acid | | | | | | | |
| Ruxolitinib | 1.98 | 1.98 | 1.98 | 1.98 | 1.98 | 1.98 | 1.98 |
| phosphate | | | | | | | |
| Phenoxyethanol | 0.5 | 0.50 | 0.50 | 0.50 | 0.50 | 0.50 | 0.50 |
| Total | 100.00 | 100.00 | 100.00 | 100.00 | 100.00 | 100.00 | 100.00 |

*The solubility of ruxolitinib phosphate has not yet been confirmed in these systems.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

1. A topical skin formulation, wherein the topical formulation is an oil-in-water emulsion, comprising:
about 0.5% to about 1.5% of a pharmaceutically acceptable salt of ruxolitinib on a free base basis by weight of the formulation;
an aqueous component;
an oil component
an non-ionic emulsifier component; and
a pharmaceutically acceptable acid.

2. The topical skin formulation of claim 1, wherein the pharmaceutically acceptable acid is selected from phosphoric acid, citric acid, and lactic acid.

3. The topical skin formulation of claim 1, wherein the pharmaceutically acceptable acid is phosphoric acid.

4. The topical skin formulation according to claim 3, wherein the phosphoric acid is present in an amount of about 0.050.1% to about 0.5% by weight of the formulation.

5. (canceled)

6. The topical skin formulation according to claim 3, wherein the phosphoric acid is present in an amount of about 0.15% to about 0.25% by weight of the formulation.

7. The topical skin formulation of claim 1, wherein the pharmaceutically acceptable acid is citric acid or lactic acid.

8. The topical skin formulation according to claim 7, wherein the citric acid or lactic acid is present in an amount of about 1% to about 5% by weight of the formulation.

9. The topical skin formulation according to claim 1, wherein the oil component comprises from about 10% to about 30% by weight of the formulation.

10. (canceled)

11. (canceled)

12. The topical skin formulation according to claim 1, wherein the oil component comprises one or more substances independently selected from petrolatums, fatty alcohols, mineral oils, triglycerides, and silicone oils.

13. The topical skin formulation according to claim 1, wherein the oil component comprises white petrolatum, cetyl alcohol, stearyl alcohol, light mineral oil, medium chain triglycerides, and dimethicone 350.

14. The topical skin formulation according to claim 1, wherein the non-ionic emulsifier component is present in an amount of about 2% to about 6% by weight of the formulation.

15. (canceled)

16. The topical skin formulation according to claim 1, wherein the non-ionic emulsifier component comprises one or more glyceryl fatty esters and one or more sorbitan fatty esters.

17. The topical skin formulation according to claim 1, wherein the non-ionic emulsifier component comprises glyceryl stearate, and polysorbate 20.

18. The topical skin formulation according to claim 1, wherein the aqueous component comprises from about 40% to about 90% by weight of the formulation.

19. (canceled)

20. The topical skin formulation according to claim 1, wherein the aqueous component comprises water and one or more substances independently selected from C₁₋₃ alkyl parabens, alkylene glycols, polyalkylene glycols; polysaccharides, and chelating agents.

21. (canceled)

22. The topical skin formulation according to claim 1, wherein the aqueous component comprises water, methyl paraben, propyl paraben, propylene glycol, polyethylene glycol 200, xanthan gum, and edetate disodium.

23. The topical skin formulation according to claim 1, wherein the water comprises from about 40% to about 60% of water by weight of the formulation.

24. (canceled)

25. (canceled)

26. The topical skin formulation according to claim 1, wherein the oil component comprises, by weight of the formulation:
about 4% of light mineral oil;
about 7% of white petrolatum;
about 3% of cetyl alcohol;
about 1.75% of stearyl alcohol;
about 1% of dimethicone 350; and
about 5% of medium chain trigylcerides.

27. (canceled)

28. (canceled)

29. (canceled)

30. (canceled)

31. (canceled)

32. The topical skin formulation according to claim 1, wherein the aqueous component comprises, by weight of the formulation:
about 45% to about 50% water;
about 15% of propylene glycol;
about 7% of polyethylene glycol 200;
about 0.1% of methyl paraben;
about 0.05% of propyl paraben;
about 0.4% of xanthan gum; and
about 0.05% of edetate disodium.

33. (canceled)

34. (canceled)

35. (canceled)

36. (canceled)

37. The topical skin formulation according claim 1, wherein the pharmaceutically acceptable salt of ruxolitinib is ruxolitinib phosphate, ruxolitinib chloride, ruxolitinib mesylate, ruxolitinib maleate, ruxolitinib sulfate, ruxolitinib ediyslate, ruxolitinib napadisylate, ruxolitinib acesulfmate, ruxolitinib oxalate, ruxolitinib L-tartrate, ruxolitinib hydrobromide, and ruxolitinib hydroiodate.

38. The topical skin formulation according to claim 1, wherein the pharmaceutically acceptable salt of ruxolitinib is ruxolitinib phosphate.

39. The topical skin formulation according to claim 1, wherein the pharmaceutically acceptable salt of ruxolitinib is present in an amount of about 0.75% or about 1.5% by weight of the formulation on a free base basis.

40. (canceled)

41. The topical skin formulation according to claim 1, further comprising about 0.4% to about 0.6% of phenoxyethanol.

42. The topical skin formulation according to claim 1, wherein the topical skin formulation comprises (by weight of the formulation):
about 10% to about 30% of the oil component;
about 40% to about 90% of the aqueous component;
about 0.5% to about 1.5% of the pharmaceutically acceptable salt of ruxolitinib; and
about 0.05% to about 0.5% of phosphoric acid.

43. (canceled)

44. (canceled)

45. (canceled)

46. The topical skin formulation according to claim 1, wherein the topical skin formulation comprises (by weight of the formulation):
about 20% to about 27% of the oil component;
about 60% to about 80% of the aqueous component;
about 0.5% to about 1.5% of the pharmaceutically acceptable salt of ruxolitinib; and
about 0.1% to about 0.5% of phosphoric acid,
wherein the aqueous component comprises about 40% to about 60% of water by weight of the formulation.

47. The topical skin formulation according to claim 1, wherein the topical skin formulation comprises (by weight of the formulation):
about 20% to about 27% of the oil component;
about 60% to about 80% of the aqueous component;
about 0.5% to about 1.5% of the pharmaceutically acceptable salt of ruxolitinib; and
about 0.05% to about 0.15% of phosphoric acid,
wherein the aqueous component comprises about 40% to about 60% of water by weight of the formulation.

48. (canceled)

49. The topical skin formulation according to claim 1, wherein the pH of the formulation is about 2.6 to about 3.3.

50. The topical skin formulation according to claim 1, wherein the formulation is a cream or a solubilized cream.

51. (canceled)

52. A method of treating a skin disorder in a patient in need thereof, comprising applying the topical skin formulation according to claim 1 to an area of skin of said patient.

53. The method of claim 52, wherein the skin disorder is one or more of atopic dermatitis, psoriasis, non-segmental vitiligo, skin sensitization, skin irritation, skin rash, contact dermatitis or allergic contact sensitization, a bullous skin disorder, vulgaris (PV), bullous pemphigoid (BP), hidradenitis suppurativa, lichen planus, alopecia, alopecia areata, frontal fibrosing alopecia, lichen planopilaris, and seborrheic dermatitis.

54-67. (canceled)
